(19) United States
(12) Patent Application Publication
(10) Pub. No.: US 2025/0107528 A1
(43) Pub. Date: Apr. 3, 2025

(54) PYRAZINE COMPOUNDS FOR THE CONTROL OF INVERTEBRATE PESTS

(71) Applicant: BASF SE, Ludwigshafen (DE)

(72) Inventors: Nikolas Huwyler, Ludwigshafen (DE); Karsten Koerber, Ludwigshafen (DE); Erik Gilberg Gilberg, Koeln (DE); Julia Pedroni, Ludwigshafen (DE); Matthias Pohlman, Ludwigshafen (DE)

(21) Appl. No.: 18/684,280

(22) PCT Filed: Aug. 12, 2022
(86) PCT No.: PCT/EP2022/072642

§ 371 Date: Feb. 16, 2024

(30) Foreign Application Priority Data
Aug. 23, 2021 (EP) 21192611.8
Aug. 23, 2021 (EP) 21192612.6
Nov. 24, 2021 (EP) 21210113.3
Dec. 16, 2021 (EP) 21215023.9

(51) Int. Cl. A01N 43/60; A01N 25/02; A01N 25/08; A01P 7/04; C07D 241/24

(52) U.S. Cl. CPC A01N 43/60; A01N 25/02; A01N 25/08; A01P 7/04; C07D 241/24

(57) ABSTRACT

The invention relates to compounds of formula II wherein the variables have the meanings as defined in the specification, to compositions com-prising them, to active compound combinations comprising them, and to their use for protecting growing plants and animals from attack or infestation by invertebrate pests, furthermore, to seed comprising such compounds.

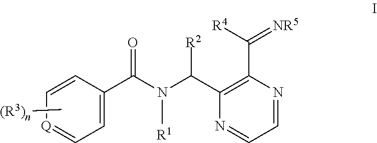

The invention relates to compounds of formula I

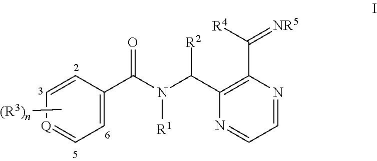

wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₂-C₄-alkenyl, C₂-C₄-alkynyl, C₁-C₄-alkyl-C₃-C₆-cycloalkyl, C₁-C₄-alkyl-C₃-C₆-halocycloalkyl, which groups are unsubstituted, or partially or fully substituted with R¹¹; or C(═N—R¹¹)R¹², C(O)R^{11a};
   R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl which ring is unsubstituted or substituted with 1 or 2 halogen; 3- to 6-membered heterocyclyl, 5- or 6-membered hetaryl, phenyl, or phenoxy, naphthyl, naphthoxy, which rings are unsubstituted or substituted with R^{3a};
   R^{11a} is NR¹²R¹³, C(O)NH₂, C(S)NH₂, C(O)OH, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with halogen, C₁-C₃-haloalkyl, and/or CN;
   R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl, S(O)ₘ—NR¹²¹R¹³¹; 3- to 6-membered heterocyclyl, 5- or 6-membered hetaryl, phenyl, phenoxy, phenyl-C₁-C₄-alkyl, naphthyl, naphthoxy, which rings are unsubstituted or substituted with R^{3a}; or
   R¹² and R¹³, together with the nitrogen atom to which they are bound, form a 3-, 4-, 5-, 6- or 7-membered saturated, partially unsaturated or fully unsaturated heterocycle, which heterocycle may additionally contain 1 or 2 heteroatoms or heteroatom groups selected from N, O, and S(O)ₘ as ring members, and which heterocycle is unsubstituted or substituted with one or more substituents selected from halogen, C₁-C₄-alkyl, C₁-C₄-haloalkyl, C₁-C₄-alkoxy, C₁-C₄-haloalkoxy, and oxo;
   R¹²¹ and R¹³¹ are independently from each other hydrogen, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl or 5- or 6-membered hetaryl which rings are unsubstituted or substituted with R^{3a}; or
   R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with halogen, C₁-C₃-haloalkyl, and/or CN;
   m is 0, 1, or 2;
   R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R^{3a};
R² is CN, C₁-C₃-alkyl, C₁-C₃-haloalkyl, C₂-C₃-alkynyl;
R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; which are unsubstituted or substituted with R^{3a};
   R^{3a} halogen, CN, NO₂, OR¹⁵, C₁-C₄-alkyl, C₁-C₄-haloalkyl, C₁-C₄-alkoxy-C₁-C₄-alkyl, C₃-C₆-cycloalkyl, C₃-C₆-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, phenyl, phenyl-C₁-C₄-alkyl, which rings are unsubstituted or substituted with halogen, CN, NO₂, C₁-C₄-alkyl, C₁-C₄-haloalkyl, C₁-C₄-alkoxy-C₁-C₄-alkyl, C₁-C₄-alkoxy, C₁-C₄-haloalkoxy,
      R^{3a} as substituent of cycloalkyl or heterocyclyl may also denote oxo;
n is 0, 1, 2, or 3;
Q is CH, CR³, or N;
R⁴ is H, OH, CN, C₁-C₄-alkyl, C₃-C₆-cycloalkyl, C₁-C₆-haloalkyl, C₁-C₆-halocycloalkyl, C₂-C₄-alkenyl, C₂-C₄-haloalkenyl, C₂-C₄-alkynyl, C₁-C₄-alkoxy, C₁-C₄-haloalkoxy, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, NR¹²R¹³, C(O)NR¹²R¹³, C(O)OR¹⁴, 3- to 6-membered heterocyclyl, 5- or 6-membered hetaryl, or phenyl, which rings are unsubstituted or substituted with R³;
R⁵ is H, OR¹⁵, NR¹²R¹³, or C₁-C₆-alkyl which is unsubstituted, or partially or fully substituted with R¹¹;
   R¹⁵ is H, C₁-C₄-alkyl, or C₁-C₄-haloalkyl, C₃-C₇-cycloalkyl, C₃-C₇-halocycloalkyl, C₃-C₇-cycloalkenyl, C₃-C₇-halocycloalkenyl, which carbon chains or rings are unsubstituted or partially or fully substituted with R¹¹; or 3- to 6-membered heterocyclyl, 5- or 6-membered hetaryl, or phenyl, which rings are unsubstituted or substituted with R³;
and the N-oxides, stereoisomers and agriculturally or veterinarily acceptable salts thereof.

The invention also provides agricultural compositions comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO 2017/192385, WO2020/070049, WO2020/201079, WO2020/201398, WO2020/208036, WO2021/037614, WO2021/122656, WO2021/068179, and WO2021/069575 describe structurally closely related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

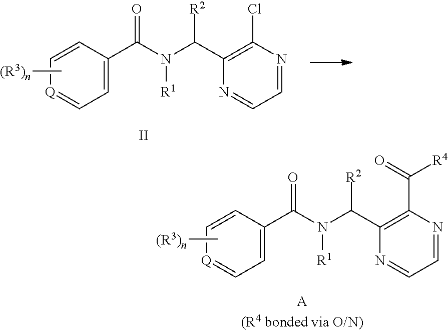

Compounds A wherein R⁴ is bound via O or N (compounds of formula A.a) can be prepared from the corresponding compounds II by a carbonylation reaction, using 0.01 to 0.2 equivalents of a palladium complex, such as palladium diacetate, bis(benzonitrile)dichloropalladium, bis(dibenzylideneacetone)palladium, or tris(dibenzylideneacetone)dipalladium, and 0.01 to 0.2 equivalents of a ligand, such as triphenylphosphine, 1,1-bis(diphenylphosphino)ferrocene, or 1,3-bis(diphenylphosphino)propane, as catalysts, in the presence of 1 to 5 equivalents of a suitable nucleophilic reagent, such as an alcohol or a primary or a secondary amine, in an inert solvent such as toluene, xylenes, mesitylene, MeCN, tetrahydrofurane (THF), 1,4-dioxane, dimethylsulfoxide (DMSO), or optionally using the nucleophilic reagent as the solvent, and optionally in the presence of additives such as triethylamine, diisopropylethylamine, NaOAc, KOAc, CsOAc, Na₂CO₃, K₂CO₃, Cs₂CO₃, NaHCO₃, KHCO₃, and/or molecular sieves, at temperatures between 25° C. and 180° C., preferably between 25° C. and the boiling point of the solvent, and in the presence of carbon monoxide gas at pressures between 1 and 50 bar (cf. Beller et al., Angew. Chem. Int. Ed. 2009, 4114 (and references therein); Takeuchi et al., J. Mol. Catal. 1991, 277). In addition, using similar catalysts and additives as above, compounds I wherein X is 0 and R⁴ is C-bound can be obtained from the corresponding compounds II by Stille reactions with alkoxyalkenylstannanes and subsequent enol ether hydrolysis (cf. H. Lin et al., Bioorg Med Chem Lett 2010, 679; Eastwood et al., Bioorg Med Chem Lett 2010, 1697). In compounds A the variables are as defined for formula I. Compounds II are known from WO2020070049.

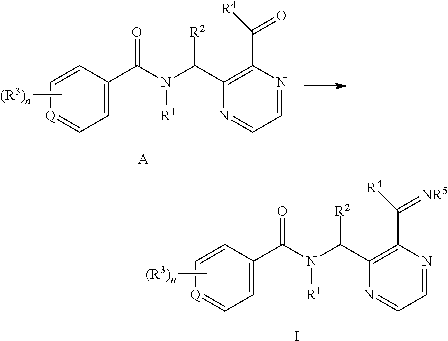

Compounds I can be obtained by condensation of a compound A, with an amine, hydroxylamine, hydrazine, or derivatives thereof, in a solvent such as (or solvent mixture composed of) dichloromethane (DCM), chloroform, benzene, toluene, xylenes, mesitylene, 1,4-dioxane, methanol, ethanol, 2-propanol, and/or water, at temperatures from 0° C. to the boiling point of the solvent or solvent mixture, and optionally with concomitant water removal, e.g. by using a Dean-Stark apparatus, or molecular sieves, or salts such as MgSO₄ or Na₂SO₄, and optionally in the presence of 0.05 to 1 equivalent of an additive such as formic acid, acetic acid, hydrochloric acid, p-toluenesulfonic acid, NaOAc, KOAc, NaOH, or KOH (cf. G. Heinisch et al., Heterocycles 1996, 151; J. Liu, Bioorg Med Chem 2008, 1096, provided that R⁴ is not 0- or N-bound).

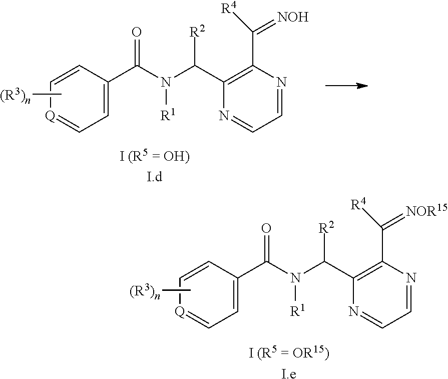

In general, compounds I wherein R⁵ is OH (compounds of formula I.d) can be converted to the corresponding compounds I wherein R⁵ is OR¹⁵ (compounds of formula I.e) with alkylating reagents such as alkyl halides, alkyl tosylates, or alkyl mesylates and following procedures known from literature (cf. Khomutov et al., Amino Acids 2010, 38, 509; Abele et al., Synthetic Communications 1998, 28, 2621; Kocak et al., Synthetic Communications 2007, 37, 1155).

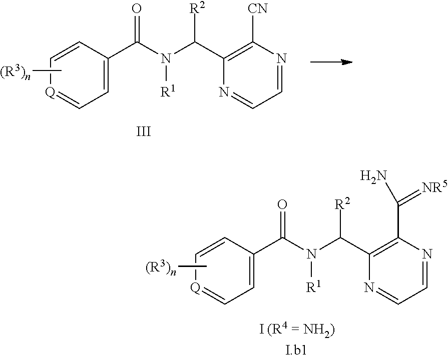

Compounds I wherein R⁴ is NH₂ (compounds of formula I.b1) can be obtained from compounds Ill by addition of a suitable nucleophile, such as hydroxylamine, hydrazine, or a derivative thereof, optionally in the presence of 1 to 2 equivalents of a base such as NaOH, KOH, Na₂CO₃, K₂CO₃, or Cs₂CO₃, and in a solvent such as (or solvent mixture composed of) methanol, ethanol, 2-propanol, and/or water, and at temperatures ranging from 0° C. to the boiling point of the solvent or solvent mixture (cf. Tiemann, Chem. Ber. 1884, 17, 126; Gobis et al., Acta Pol Pharm (Drug Research) 2006, 39).

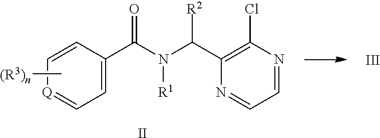

Compounds III can be obtained by reaction of compounds II with 1 to 2 equivalents of Zn(CN)₂ in the presence of 0.01 to 0.2 equivalents of a transition-metal complex, such as palladium diacetate, palladium trifluoroacetate, tris(dibenzylideneacetone)dipalladium, tetrakis(triphenylphosphine)palladium, or nickel dichloride, and 0.01 to 0.2 equivalents of a ligand, such as 2-[di(tertbutyl)phosphino]-1,1′-binaphthyl, 1,1-bis(diphenylphosphino)ferrocene, or BINAP, as catalysts, and 0.1 to 0.5 equivalents of zinc powder or zinc flakes as a cocatalyst, and optionally with 1 to 3 equivalents of a base, such as 4-(dimethylamino)pyridine or sodium tert-butoxide, in a solvent such as dimethylformamide (DMF), N,N-dimethylacetamide (DMA), or acetonitrile, at temperatures between 25° C. and 150° C., preferably between 25° C. and the boiling point of the solvent (cf. A. Littke et al., Org. Lett. 2007, 1711).

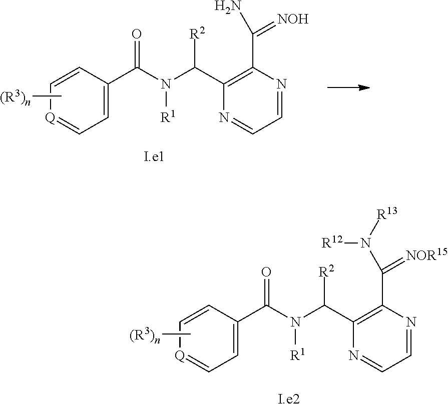

Compounds I wherein R⁵ is OR¹⁵ and R⁴ is NH₂ (compounds I.e1) can be converted to compounds I wherein R⁵ is OR¹⁵ and R⁴ is NR¹²R¹³ (compounds i.e2) in two steps by reaction with sodium nitrite in aqueous HCl followed by substitution of the chloride with HNR¹²R¹³ (cf. K. Gobis et al., Acta Pol Pharm (Drug Research) 2006, 39; A. M. Martsynkevich et al., Russ. Chem. Bull. Int. Ed. 2011, 521).

If individual compounds I cannot be obtained by the routes described above, they can be prepared by derivatization of other compounds I.

The reaction mixtures are worked up in a customary manner, for example by mixing with water, extracting with an appropriate organic solvent, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the pest to be controlled.

The organic moieties groups mentioned in the above definitions of the variables are—like the term halogen—collective terms for individual listings of the individual group members. The prefix Cₙ-Cₘ indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, npentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluoromethoxy, 2,2,2-trichloroethoxy, pentafluoroethoxy and the like.

The term “alkylthio “(alkylsulfanyl: —S-alkyl)” as used herein refers to a straight-chain or branched saturated alkyl group having 1 to 10 carbon atoms, preferably 1 to 4 carbon atoms (=C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: —S(═O)—C₁-C₆-alkyl), as used herein refers to a straightchain or branched saturated alkyl group (as mentioned above) having 1 to 10 carbon atoms, preferably 1 to 4 carbon atoms (=C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (=C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a mono- or bicyclic, preferably monocyclic, cycloaliphatic radical having usually from 3 to 10 or from 3 to 6 carbon atoms, such as cyclopropyl (cC₃H₅), cyclobutyl (cC₄H₇), cyclopentyl (cC₅H₉), cyclohexyl (cC₆H₁₁), cycloheptyl, cyclooctyl, cyclononyl, and cyclodecyl, or cyclopropyl, cyclobutyl, cyclopentyl, and cyclohexyl.

The term “halocycloalkyl” as used herein and in the halocycloalkyl moieties of halocycloalkoxy and halocycloalkylthio denotes in each case a mono- or bicyclic, preferably monocyclic, cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “cycloalkenyl” as used herein and in the cycloalkenyl moieties of cycloalkenyloxy and cycloalkenylthio denotes in each case a mono- or bicyclic, preferably monocyclic, singly unsaturated non-aromatic radical having usually from 3 to 10, e.g. 3 or 4 or from 5 to 10 carbon atoms, preferably from 3- to 8 carbon atoms. Examples are cyclopenten-1-yl, and cyclohexen-1-yl.

The term “halocycloalkenyl” as used herein and in the halocycloalkenyl moieties of halocycloalkenyloxy and halocycloalkenylthio denotes in each case a mono- or bicyclic, preferably monocyclic, singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3- to 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O, and S as ring members, wherein S-atoms as ring members may be present as S, SO, or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothiomorpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O, and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O, and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

In a preferred embodiment, the compounds I are present in form of a mixture of compounds I.A and I.B, wherein compound I.A with S-configuration of the carbon atom neighboring the nitrogen is present in an amount of more than 50% by weight, in particular of at least 70% by weight, more particularly of at least 85% by weight, specifically of at least 90% by weight, based on the total weight of compounds I.A and I.B.

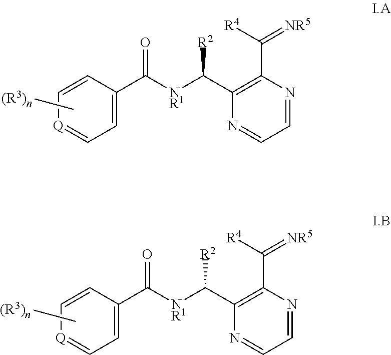

In one particularly preferred embodiment of the invention, the method comprises the step of contacting the plant, parts of it, its propagation material, the pests, their food supply, habitat or breeding grounds with a pesticidally effective amount of a compound of formula I.A.

Preferably R¹ is H, C₁-C₆-alkyl, C₃-C₄-alkenyl, C₃-C₄-alkynyl, C₃-C₆-cycloalkyl, or C₁-C₄-alkyl-C₃-C₆-cycloalkyl. Particularly R¹ is H or CH₂-cC₃H₅.

Preferably R² is CH₃.

R³ is preferably halogen, CN, C₁-C₄-haloalkyl, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl. Index m in R³ is preferably 2. Index n is preferably 2.

In one embodiment Q is CH or CR³, preferably CH. Such compounds correspond to formula I.1

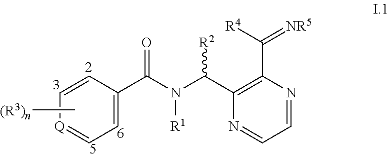

In another embodiment Q is N. Such compounds correspond to formula I.2

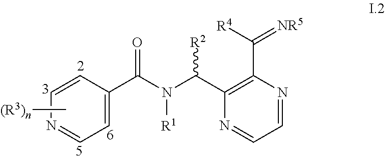

R³ groups stand preferably in positions 3 and 5.

In another embodiment R³ is preferably halogen, CN, C₁-C₄-haloalkyl, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-Cycloalkyl, S(O)ₘ—C₃-C₄-halocycloalkyl, or
S(O)ₘ—R¹⁴, wherein R¹⁴ is phenyl, which is partially substituted with R^{3a}.

In another embodiment of formula I compounds R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; wherein rings are unsubstituted or substituted with R¹¹.

In another embodiment of formula I compounds R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; wherein rings are unsubstituted or substituted with one or more CN, OH, C₁-C₄-alkoxy.

In another embodiment of formula I compounds R³ is C₃-C₆-cycloalkyl unsubstituted or substituted with one or more CN, OH, C₁-C₄-alkoxy.

R⁴ is preferably H, C₁-C₃-alkyl, or C₁-C₃-haloalkyl.

In another embodiment R⁴ is H, C₁-C₃-alkyl, C₁-C₃-haloalkyl, or NR¹²R¹³ wherein R¹² and R¹³ are independently from each other H or C₁-C₃-alkyl.

R⁵ is preferably OH, C₁-C₃-alkoxy, or C₁-C₃-haloalkoxy.

In a preferred embodiment R⁵ is OH, C₁-C₃-alkoxy, C₁-C₃-haloalkoxy, or NR¹²R¹³, wherein R¹² and R¹³ are independently from each other H or C(O)NR¹²¹R¹³¹, with R¹²¹, and R¹³¹ being preferably independently from each other H, C₁-C₃-alkyl, or C₃-C₄-cycloalkyl-C₁-C₂-alkyl.

In another preferred embodiment R⁵ is NR¹²R¹³, wherein R¹² and R¹³ form, together with the N atom they are bound to, a 4- to 7-membered heterocycle unsubstituted or substituted with R^{3a} wherein R^{3a} is preferably halogen, C₁-C₄-alkyl, C₁-C₄-haloalkyl, or oxo.

R⁵ is present as E-/Z-isomers of formula I. The E-isomer is preferred, particularly in formula I compounds wherein R⁵ is OR¹⁵ and R⁴ is H or alkyl, such as CH₃.

In particular with a view to their use, preference is given to the compounds of formula I compiled in the tables below, which compounds correspond to formula I.1*, and I.2*, resp. Each of the groups mentioned for a substituent in the tables is furthermore per se, independently of the combination in which it is mentioned, a particularly preferred aspect of the substituent in question.

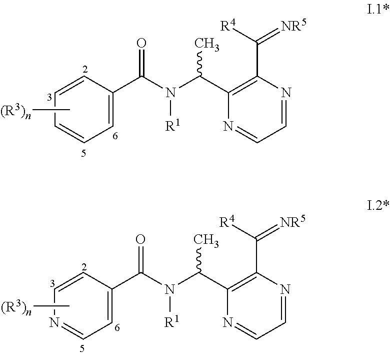

### Table 1

Compounds of formula I.1* in which R¹ is H, R⁴ is H, and the combination of NR⁵ and (R³)ₙ for a compound corresponds in each case to one row of Table A

### Table 2

Compounds of formula I.1* in which R¹ is CH₃, R⁴ is H, and the combination of NR⁵ and (R³)ₙ for a compound corresponds in each case to one row of Table A

### Table 3

Compounds of formula I.1* in which R¹ is C₂H₅, R⁴ is H, and the combination of NR⁵ and (R³)ₙ for a compound corresponds in each case to one row of Table A

### Table 4

Compounds of formula I.1* in which R¹ is CH₂-cC₃H₅, R⁴ is H, and the combination of NR⁵ and (R³)ₙ for a compound corresponds in each case to one row of Table A

### Table 5

Compounds of formula I.1* in which R¹ is CH₂-cC₅H₉, R⁴ is H, and the combination of NR⁵ and (R³)ₙ for a compound corresponds in each case to one row of Table A

### Table 6

Compounds of formula I.1* in which R¹ is CH₂CH═CH₂, R⁴ is H, and the combination of NR⁵ and (R³)ₙ for a compound corresponds in each case to one row of Table A

### Table 7

Compounds of formula I.1* in which R¹ is CH₂C≡CH, R⁴ is H, and the combination of NR⁵ and (R³)ₙ for a compound corresponds in each case to one row of Table A

### Table 8

Compounds of formula I.1* in which R¹ is H, R⁴ is CN, and the combination of NR⁵ and (R³)ₙ for a compound corresponds in each case to one row of Table A

### Table 9

Compounds of formula I.1* in which R¹ is CH₃, R⁴ is CN, and the combination of NR⁵ and (R³)ₙ for a compound corresponds in each case to one row of Table A

### Table 10

Compounds of formula I.1* in which R¹ is C₂H₅, R⁴ is CN, and the combination of NR⁵ and (R³)ₙ for a compound corresponds in each case to one row of Table A

### Table 11

Compounds of formula I.1* in which R¹ is CH₂-cC₃H₅, R⁴ is CN, and the combination of NR⁵ and (R³)ₙ for a compound corresponds in each case to one row of Table A

### Table 12

Compounds of formula I.1* in which R¹ is CH₂-cC₅H₉, R⁴ is CN, and the combination of NR⁵ and (R³)ₙ for a compound corresponds in each case to one row of Table A

### Table 13

Compounds of formula I.1* in which R¹ is CH₂CH═CH₂, R⁴ is CN, and the combination of NR⁵ and (R³)ₙ for a compound corresponds in each case to one row of Table A

### Table 14

Compounds of formula I.1* in which R¹ is CH₂C≡CH, R⁴ is CN, and the combination of NR⁵ and (R³)ₙ for a compound corresponds in each case to one row of Table A

### Table 15

Compounds of formula I.1* in which R¹ is H, R⁴ is CH₃, and the combination of NR⁵ and (R³)ₙ for a compound corresponds in each case to one row of Table A

### Table 16

Compounds of formula I.1* in which R¹ is CH₃, R⁴ is CH₃, and the combination of NR⁵ and (R³)ₙ for a compound corresponds in each case to one row of Table A

### Table 17

Compounds of formula I.1* in which R¹ is C₂H₅, R⁴ is CH₃, and the combination of NR⁵ and (R³)ₙ for a compound corresponds in each case to one row of Table A

### Table 18

Compounds of formula I.1* in which R¹ is CH₂-cC₃H₅, R⁴ is CH₃, and the combination of NR⁵ and (R³)ₙ for a compound corresponds in each case to one row of Table A

### Table 19

Compounds of formula I.1* in which R¹ is CH₂-cC₅H₉, R⁴ is CH₃, and the combination of NR⁵ and (R³)ₙ for a compound corresponds in each case to one row of Table A

### Table 20

Compounds of formula I.1* in which R¹ is CH₂CH═CH₂, R⁴ is CH₃, and the combination of NR⁵ and (R³)ₙ for a compound corresponds in each case to one row of Table A

### Table 21

Compounds of formula I.1* in which R¹ is CH₂C≡CH, R⁴ is CH₃, and the combination of NR⁵ and (R³)ₙ for a compound corresponds in each case to one row of Table A

### Table 22

Compounds of formula I.1* in which R¹ is H, R⁴ is C₂H₅, and the combination of NR⁵ and (R³)ₙ for a compound corresponds in each case to one row of Table A

### Table 23

Compounds of formula I.1* in which R¹ is CH₃, R⁴ is C₂H₅, and the combination of NR⁵ and (R³)ₙ for a compound corresponds in each case to one row of Table A

### Table 24

Compounds of formula I.1* in which R¹ is C₂H₅, R⁴ is C₂H₅, and the combination of NR⁵ and (R³)ₙ for a compound corresponds in each case to one row of Table A

### Table 25

Compounds of formula I.1* in which R¹ is CH₂-cC₃H₅, R⁴ is C₂H₅, and the combination of NR⁵ and (R³)ₙ for a compound corresponds in each case to one row of Table A

### Table 26

Compounds of formula I.1* in which R¹ is CH₂-cC₅H₉, R⁴ is C₂H₅, and the combination of NR⁵ and (R³)ₙ for a compound corresponds in each case to one row of Table A

### Table 27

Compounds of formula I.1* in which R¹ is CH₂CH═CH₂, R⁴ is C₂H₅, and the combination of NR⁵ and (R³)ₙ for a compound corresponds in each case to one row of Table A

### Table 28

Compounds of formula I.1* in which R¹ is CH₂C≡CH, R⁴ is C₂H₅, and the combination of NR⁵ and (R³)ₙ for a compound corresponds in each case to one row of Table A

### Table 29

Compounds of formula I.1* in which R¹ is H, R⁴ is CH₂CH₂CH₃, and the combination of NR⁵ and (R³)ₙ for a compound corresponds in each case to one row of Table A

### Table 30

Compounds of formula I.1* in which R¹ is CH₃, R⁴ is CH₂CH₂CH₃, and the combination of NR⁵ and (R³)ₙ for a compound corresponds in each case to one row of Table A

### Table 31

Compounds of formula I.1* in which R¹ is C₂H₅, R⁴ is CH₂CH₂CH₃, and the combination of NR⁵ and (R³)ₙ for a compound corresponds in each case to one row of Table A

### Table 32

Compounds of formula I.1* in which R¹ is CH₂-cC₃H₅, R⁴ is CH₂CH₂CH₃, and the combination of X and (R³)ₙ for a compound corresponds in each case to one row of Table A

### Table 33

Compounds of formula I.1* in which R¹ is CH₂-cC₅H₉, R⁴ is CH₂CH₂CH₃, and the combination of NR⁵ and (R³)ₙ for a compound corresponds in each case to one row of Table A

### Table 34

Compounds of formula I.1* in which R¹ is CH₂CH═CH₂, R⁴ is CH₂CH₂CH₃, and the combination of NR⁵ and (R³)ₙ for a compound corresponds in each case to one row of Table A

### Table 35

Compounds of formula I.1* in which R¹ is CH₂C≡CH, R⁴ is CH₂CH₂CH₃, and the combination of NR⁵ and (R³)ₙ for a compound corresponds in each case to one row of Table A

### Table 36

Compounds of formula I.1* in which R¹ is H, R⁴ is CH(CH₃)₂, and the combination of NR⁵ and (R³)ₙ for a compound corresponds in each case to one row of Table A

### Table 37

Compounds of formula I.1* in which R¹ is CH₃, R⁴ is CH(CH₃)₂, and the combination of NR⁵ and (R³)ₙ for a compound corresponds in each case to one row of Table A

### Table 38

Compounds of formula I.1* in which R¹ is C₂H₅, R⁴ is CH(CH₃)₂, and the combination of NR⁵ and (R³)ₙ for a compound corresponds in each case to one row of Table A

### Table 39

Compounds of formula I.1* in which R¹ is CH₂-cC₃H₅, R⁴ is CH(CH₃)₂, and the combination of NR⁵ and (R³)ₙ for a compound corresponds in each case to one row of Table A

### Table 40

Compounds of formula I.1* in which R¹ is CH₂-cC₅H₉, R⁴ is CH(CH₃)₂, and the combination of NR⁵ and (R³)ₙ for a compound corresponds in each case to one row of Table A

### Table 41

Compounds of formula I.1* in which R¹ is CH₂CH═CH₂, R⁴ is CH(CH₃)₂, and the combination of NR⁵ and (R³)ₙ for a compound corresponds in each case to one row of Table A

### Table 42

Compounds of formula I.1* in which R¹ is CH₂C≡CH, R⁴ is CH(CH₃)₂, and the combination of NR⁵ and (R³)ₙ for a compound corresponds in each case to one row of Table A

### Table 43

Compounds of formula I.1* in which R¹ is H, R⁴ is C(═O)NH₂, and the combination of NR⁵ and (R³)ₙ for a compound corresponds in each case to one row of Table A

### Table 44

Compounds of formula I.1* in which R¹ is CH₃, R⁴ is C(═O)NH₂, and the combination of NR⁵ and (R³)ₙ for a compound corresponds in each case to one row of Table A

### Table 45

Compounds of formula I.1* in which R¹ is C₂H₅, R⁴ is C(═O)NH₂, and the combination of NR⁵ and (R³)ₙ for a compound corresponds in each case to one row of Table A

### Table 46

Compounds of formula I.1* in which R¹ is CH₂-cC₃H₅, R⁴ is C(═O)NH₂, and the combination of NR⁵ and (R³)ₙ for a compound corresponds in each case to one row of Table A

### Table 47

Compounds of formula I.1* in which R¹ is CH₂-cC₅H₉, R⁴ is C(═O)NH₂, and the combination of NR⁵ and (R³)ₙ for a compound corresponds in each case to one row of Table A

### Table 48

Compounds of formula I.1* in which R¹ is CH₂CH═CH₂, R⁴ is C(═O)NH₂, and the combination of NR⁵ and (R³)ₙ for a compound corresponds in each case to one row of Table A

### Table 49

Compounds of formula I.1* in which R¹ is CH₂C≡CH, R⁴ is C(═O)NH₂, and the combination of NR⁵ and (R³)ₙ for a compound corresponds in each case to one row of Table A

### Table 50

Compounds of formula I.1* in which R¹ is H, R⁴ is C(═O)NHCH₃, and the combination of NR⁵ and (R³)ₙ for a compound corresponds in each case to one row of Table A

### Table 51

Compounds of formula I.1* in which R¹ is CH₃, R⁴ is C(═O)NHCH₃, and the combination of NR⁵ and (R³)ₙ for a compound corresponds in each case to one row of Table A

### Table 52

Compounds of formula I.1* in which R¹ is C₂H₅, R⁴ is C(═O)NHCH₃, and the combination of NR⁵ and (R³)ₙ for a compound corresponds in each case to one row of Table A

### Table 53

Compounds of formula I.1* in which R¹ is CH₂-cC₃H₅, R⁴ is C(═O)NHCH₃, and the combination of NR⁵ and (R³)ₙ for a compound corresponds in each case to one row of Table A

### Table 54

Compounds of formula I.1* in which R¹ is CH₂-cC₅H₉, R⁴ is C(═O)NHCH₃, and the combination of NR⁵ and (R³)ₙ for a compound corresponds in each case to one row of Table A

### Table 55

Compounds of formula I.1* in which R¹ is CH₂CH═CH₂, R⁴ is C(═O)NHCH₃, and the combination of NR⁵ and (R³)ₙ for a compound corresponds in each case to one row of Table A

### Table 56

Compounds of formula I.1* in which R¹ is CH₂C≡CH, R⁴ is C(═O)NHCH₃, and the combination of NR⁵ and (R³)ₙ for a compound corresponds in each case to one row of Table A

### Table 57

Compounds of formula I.1* in which R¹ is H, R⁴ is C(═O)NHC₂H₅, and the combination of NR⁵ and (R³)ₙ for a compound corresponds in each case to one row of Table A

### Table 58

Compounds of formula I.1* in which R¹ is CH₃, R⁴ is C(═O)NHC₂H₅, and the combination of NR⁵ and (R³)ₙ for a compound corresponds in each case to one row of Table A

### Table 59

Compounds of formula I.1* in which R¹ is C₂H₅, R⁴ is C(═O)NHC₂H₅, and the combination of NR⁵ and (R³)ₙ for a compound corresponds in each case to one row of Table A

### Table 60

Compounds of formula I.1* in which R¹ is CH₂-cC₃H₅, R⁴ is C(═O)NHC₂H₅, and the combination of NR⁵ and (R³)ₙ for a compound corresponds in each case to one row of Table A

### Table 61

Compounds of formula I.1* in which R¹ is CH₂-cC₅H₉, R⁴ is C(═O)NHC₂H₅, and the combination of NR⁵ and (R³)ₙ for a compound corresponds in each case to one row of Table A

### Table 62

Compounds of formula I.1* in which R¹ is CH₂CH═CH₂, R⁴ is C(═O)NHC₂H₅, and the combination of NR⁵ and (R³)ₙ for a compound corresponds in each case to one row of Table A

### Table 63

Compounds of formula I.1* in which R¹ is CH₂C≡CH, R⁴ is C(═O)NHC₂H₅, and the combination of NR⁵ and (R³)ₙ for a compound corresponds in each case to one row of Table A

### Table 64

Compounds of formula I.1* in which R¹ is H, R⁴ is C(═O)N(CH₃)₂, and the combination of NR⁵ and (R³)ₙ for a compound corresponds in each case to one row of Table A

### Table 65

Compounds of formula I.1* in which R¹ is CH₃, R⁴ is C(═O)N(CH₃)₂, and the combination of NR⁵ and (R³)ₙ for a compound corresponds in each case to one row of Table A

### Table 66

Compounds of formula I.1* in which R¹ is C₂H₅, R⁴ is C(═O)N(CH₃)₂, and the combination of NR⁵ and (R³)ₙ for a compound corresponds in each case to one row of Table A

### Table 67

Compounds of formula I.1* in which R¹ is CH₂-cC₃H₅, R⁴ is C(═O)N(CH₃)₂, and the combination of NR⁵ and (R³)ₙ for a compound corresponds in each case to one row of Table A

### Table 68

Compounds of formula I.1* in which R¹ is CH₂-cC₅H₉, R⁴ is C(═O)N(CH₃)₂, and the combination of NR⁵ and (R³)ₙ for a compound corresponds in each case to one row of Table A

### Table 69

Compounds of formula I.1* in which R¹ is CH₂CH═CH₂, R⁴ is C(═O)N(CH₃)₂, and the combination of NR⁵ and (R³)ₙ for a compound corresponds in each case to one row of Table A

### Table 70

Compounds of formula I.1* in which R¹ is CH₂C≡CH, R⁴ is C(═O)N(CH₃)₂, and the combination of NR⁵ and (R³)ₙ for a compound corresponds in each case to one row of Table A

### Table 71

Compounds of formula I.1* in which R¹ is H, R⁴ is C(═O)N(CH₃)C₂H₅, and the combination of NR⁵ and (R³)ₙ for a compound corresponds in each case to one row of Table A

### Table 72

Compounds of formula I.1* in which R¹ is CH₃, R⁴ is C(═O)N(CH₃)C₂H₅, and the combination of NR⁵ and (R³)ₙ for a compound corresponds in each case to one row of Table A

### Table 73

Compounds of formula I.1* in which R¹ is C₂H₅, R⁴ is C(═O)N(CH₃)C₂H₅, and the combination of NR⁵ and (R³)ₙ for a compound corresponds in each case to one row of Table A

### Table 74

Compounds of formula I.1* in which R¹ is CH₂-cC₃H₅, R⁴ is C(═O)N(CH₃)C₂H₅, and the combination of NR⁵ and (R³)ₙ for a compound corresponds in each case to one row of Table A

### Table 75

Compounds of formula I.1* in which R¹ is CH₂-cC₅H₉, R⁴ is C(═O)N(CH₃)C₂H₅, and the combination of NR⁵ and (R³)ₙ for a compound corresponds in each case to one row of Table A

### Table 76

Compounds of formula I.1* in which R¹ is CH₂CH═CH₂, R⁴ is C(═O)N(CH₃)C₂H₅, and the combination of NR⁵ and (R³)ₙ for a compound corresponds in each case to one row of Table A

### Table 77

Compounds of formula I.1* in which R¹ is CH₂C≡CH, R⁴ is C(═O)N(CH₃)C₂H₅, and the combination of NR⁵ and (R³)ₙ for a compound corresponds in each case to one row of Table A

### Table 78

Compounds of formula I.1* in which R¹ is H, R⁴ is C(═O)OCH₃, and the combination of NR⁵ and (R³)ₙ for a compound corresponds in each case to one row of Table A

### Table 79

Compounds of formula I.1* in which R¹ is CH₃, R⁴ is C(═O)OCH₃, and the combination of NR⁵ and (R³)ₙ for a compound corresponds in each case to one row of Table A

### Table 80

Compounds of formula I.1* in which R¹ is C₂H₅, R⁴ is C(═O)OCH₃, and the combination of NR⁵ and (R³)ₙ for a compound corresponds in each case to one row of Table A

### Table 81

Compounds of formula I.1* in which R¹ is CH₂-cC₃H₅, R⁴ is C(═O)OCH₃, and the combination of NR⁵ and (R³)ₙ for a compound corresponds in each case to one row of Table A

### Table 82

Compounds of formula I.1* in which R¹ is CH₂-cC₅H₉, R⁴ is C(═O)OCH₃, and the combination of NR⁵ and (R³)ₙ for a compound corresponds in each case to one row of Table A

### Table 83

Compounds of formula I.1* in which R¹ is CH₂CH═CH₂, R⁴ is C(═O)OCH₃, and the combination of NR⁵ and (R³)ₙ for a compound corresponds in each case to one row of Table A

### Table 84

Compounds of formula I.1* in which R¹ is CH₂C≡CH, R⁴ is C(═O)OCH₃, and the combination of NR⁵ and (R³)ₙ for a compound corresponds in each case to one row of Table A

### Table 85

Compounds of formula I.1* in which R¹ is H, R⁴ is C(═O)OC₂H₅, and the combination of NR⁵ and (R³)ₙ for a compound corresponds in each case to one row of Table A

### Table 86

Compounds of formula I.1* in which R¹ is CH₃, R⁴ is C(═O)OC₂H₅, and the combination of NR⁵ and (R³)ₙ for a compound corresponds in each case to one row of Table A

### Table 87

Compounds of formula I.1* in which R¹ is C₂H₅, R⁴ is C(═O)OC₂H₅, and the combination of NR⁵ and (R³)ₙ for a compound corresponds in each case to one row of Table A

### Table 88

Compounds of formula I.1* in which R¹ is CH₂-cC₃H₅, R⁴ is C(═O)OC₂H₅, and the combination of NR⁵ and (R³)ₙ for a compound corresponds in each case to one row of Table A

### Table 89

Compounds of formula I.1* in which R¹ is CH₂-cC₅H₉, R⁴ is C(═O)OC₂H₅, and the combination of NR⁵ and (R³)ₙ for a compound corresponds in each case to one row of Table A

### Table 90

Compounds of formula I.1* in which R¹ is CH₂CH═CH₂, R⁴ is C(═O)OC₂H₅, and the combination of NR⁵ and (R³)ₙ for a compound corresponds in each case to one row of Table A

### Table 91

Compounds of formula I.1* in which R¹ is CH₂C≡CH, R⁴ is C(═O)OC₂H₅, and the combination of NR⁵ and (R³)ₙ for a compound corresponds in each case to one row of Table A

### Table 92

Compounds of formula I.1* in which R¹ is H, R⁴ is NH₂, and the combination of NR⁵ and (R³)ₙ for a compound corresponds in each case to one row of Table A

### Table 93

Compounds of formula I.1* in which R¹ is CH₃, R⁴ is NH₂, and the combination of NR⁵ and (R³)ₙ for a compound corresponds in each case to one row of Table A

### Table 94

Compounds of formula I.1* in which R¹ is C₂H₅, R⁴ is NH₂, and the combination of NR⁵ and (R³)ₙ for a compound corresponds in each case to one row of Table A

### Table 95

Compounds of formula I.1* in which R¹ is CH₂-cC₃H₅, R⁴ is NH₂, and the combination of NR⁵ and (R³)ₙ for a compound corresponds in each case to one row of Table A

### Table 96

Compounds of formula I.1* in which R¹ is CH₂-cC₅H₉, R⁴ is NH₂, and the combination of NR⁵ and (R³)ₙ for a compound corresponds in each case to one row of Table A

### Table 97

Compounds of formula I.1* in which R¹ is CH₂CH═CH₂, R⁴ is NH₂, and the combination of NR⁵ and (R³)ₙ for a compound corresponds in each case to one row of Table A

### Table 98

Compounds of formula I.1* in which R¹ is CH₂C≡CH, R⁴ is NH₂, and the combination of NR⁵ and (R³)ₙ for a compound corresponds in each case to one row of Table A

### Table 99

Compounds of formula I.1* in which R¹ is H, R⁴ is NHCH₃, and the combination of NR⁵ and (R³)ₙ for a compound corresponds in each case to one row of Table A

### Table 100

Compounds of formula I.1* in which R¹ is CH₃, R⁴ is NHCH₃, and the combination of NR⁵ and (R³)ₙ for a compound corresponds in each case to one row of Table A

### Table 101

Compounds of formula I.1* in which R¹ is C₂H₅, R⁴ is NHCH₃, and the combination of NR⁵ and (R³)ₙ for a compound corresponds in each case to one row of Table A

### Table 102

Compounds of formula I.1* in which R¹ is CH₂-cC₃H₅, R⁴ is NHCH₃, and the combination of NR⁵ and (R³)ₙ for a compound corresponds in each case to one row of Table A

### Table 103

Compounds of formula I.1* in which R¹ is CH₂-cC₅H₉, R⁴ is NHCH₃, and the combination of NR⁵ and (R³)ₙ for a compound corresponds in each case to one row of Table A

### Table 104

Compounds of formula I.1* in which R¹ is CH₂CH═CH₂, R⁴ is NHCH₃, and the combination of NR⁵ and (R³)ₙ for a compound corresponds in each case to one row of Table A

### Table 105

Compounds of formula I.1* in which R¹ is CH₂C≡CH, R⁴ is NHCH₃, and the combination of NR⁵ and (R³)ₙ for a compound corresponds in each case to one row of Table A

### Table 106

Compounds of formula I.1* in which R¹ is H, R⁴ is N(CH₃)C₂H₅, and the combination of NR⁵ and (R³)ₙ for a compound corresponds in each case to one row of Table A

### Table 107

Compounds of formula I.1* in which R¹ is CH₃, R⁴ is N(CH₃)C₂H₅, and the combination of NR⁵ and (R³)ₙ for a compound corresponds in each case to one row of Table A

### Table 108

Compounds of formula I.1* in which R¹ is C₂H₅, R⁴ is N(CH₃)C₂H₅, and the combination of NR⁵ and (R³)ₙ for a compound corresponds in each case to one row of Table A

### Table 109

Compounds of formula I.1* in which R¹ is CH₂-cC₃H₅, R⁴ is N(CH₃)C₂H₅, and the combination of NR⁵ and (R³)ₙ for a compound corresponds in each case to one row of Table A

### Table 110

Compounds of formula I.1* in which R¹ is CH₂-cC₅H₉, R⁴ is N(CH₃)C₂H₅, and the combination of NR⁵ and (R³)ₙ for a compound corresponds in each case to one row of Table A

### Table 111

Compounds of formula I.1* in which R¹ is CH₂CH═CH₂, R⁴ is N(CH₃)C₂H₅, and the combination of NR⁵ and (R³)ₙ for a compound corresponds in each case to one row of Table A

### Table 112

Compounds of formula I.1* in which R¹ is CH₂C≡CH, R⁴ is N(CH₃)C₂H₅, and the combination of NR⁵ and (R³)ₙ for a compound corresponds in each case to one row of Table A

### Table 113

Compounds of formula I.1* in which R¹ is H, R⁴ is NHCH₂CN, and the combination of NR⁵ and (R³)ₙ for a compound corresponds in each case to one row of Table A

### Table 114

Compounds of formula I.1* in which R¹ is CH₃, R⁴ is NHCH₂CN, and the combination of NR⁵ and (R³)ₙ for a compound corresponds in each case to one row of Table A

### Table 115

Compounds of formula I.1* in which R¹ is C₂H₅, R⁴ is NHCH₂CN, and the combination of NR⁵ and (R³)ₙ for a compound corresponds in each case to one row of Table A

### Table 116

Compounds of formula I.1* in which R¹ is CH₂-cC₃H₅, R⁴ is NHCH₂CN, and the combination of NR⁵ and (R³)ₙ for a compound corresponds in each case to one row of Table A

### Table 117

Compounds of formula I.1* in which R¹ is CH₂-cC₅H₉, R⁴ is NHCH₂CN, and the combination of NR⁵ and (R³)ₙ for a compound corresponds in each case to one row of Table A

### Table 118

Compounds of formula I.1* in which R¹ is CH₂CH═CH₂, R⁴ is NHCH₂CN, and the combination of NR⁵ and (R³)ₙ for a compound corresponds in each case to one row of Table A

### Table 119

Compounds of formula I.1* in which R¹ is CH₂C≡CH, R⁴ is NHCH₂CN, and the combination of NR⁵ and (R³)ₙ for a compound corresponds in each case to one row of Table A

### Table 120

Compounds of formula I.1* in which R¹ is H, R⁴ is 4,5-dihydrooxazol-2-yl, and the combination of NR⁵ and (R³)ₙ for a compound corresponds in each case to one row of Table A

### Table 121

Compounds of formula I.1* in which R¹ is CH₃, R⁴ is 4,5-dihydrooxazol-2-yl, and the combination of NR⁵ and (R³)ₙ for a compound corresponds in each case to one row of Table A

### Table 122

Compounds of formula I.1* in which R¹ is C₂H₅, R⁴ is 4,5-dihydrooxazol-2-yl, and the combination of NR⁵ and (R³)ₙ for a compound corresponds in each case to one row of Table A

### Table 123

Compounds of formula I.1* in which R¹ is CH₂-cC₃H₅, R⁴ is 4,5-dihydrooxazol-2-yl, and the combination of NR⁵ and (R³)ₙ for a compound corresponds in each case to one row of Table A

### Table 124

Compounds of formula I.1* in which R¹ is CH₂-cC₅H₉, R⁴ is 4,5-dihydrooxazol-2-yl, and the combination of NR⁵ and (R³)ₙ for a compound corresponds in each case to one row of Table A

### Table 125

Compounds of formula I.1* in which R¹ is CH₂CH═CH₂, R⁴ is 4,5-dihydrooxazol-2-yl, and the combination of NR⁵ and (R³)ₙ for a compound corresponds in each case to one row of Table A

### Table 126

Compounds of formula I.1* in which R¹ is CH₂C≡CH, R⁴ is 4,5-dihydrooxazol-2-yl, and the combination of NR⁵ and (R³)ₙ for a compound corresponds in each case to one row of Table A

### Table 127

Compounds of formula I.2* in which R¹ is H, R⁴ is H, and the combination of NR⁵ and (R³)ₙ for a compound corresponds in each case to one row of Table A

### Table 128

Compounds of formula I.2* in which R¹ is CH₃, R⁴ is H, and the combination of NR⁵ and (R³)ₙ for a compound corresponds in each case to one row of Table A

### Table 129

Compounds of formula I.2* in which R¹ is C₂H₅, R⁴ is H, and the combination of NR⁵ and (R³)ₙ for a compound corresponds in each case to one row of Table A

### Table 130

Compounds of formula I.2* in which R¹ is CH₂-cC₃H₅, R⁴ is H, and the combination of NR⁵ and (R³)ₙ for a compound corresponds in each case to one row of Table A

### Table 131

Compounds of formula I.2* in which R¹ is CH₂-cC₅H₉, R⁴ is H, and the combination of NR⁵ and (R³)ₙ for a compound corresponds in each case to one row of Table A

### Table 132

Compounds of formula I.2* in which R¹ is CH₂CH═CH₂, R⁴ is H, and the combination of NR⁵ and (R³)ₙ for a compound corresponds in each case to one row of Table A

### Table 133

Compounds of formula I.2* in which R¹ is CH₂C≡CH, R⁴ is H, and the combination of NR⁵ and (R³)ₙ for a compound corresponds in each case to one row of Table A

### Table 134

Compounds of formula I.2* in which R¹ is H, R⁴ is CN, and the combination of NR⁵ and (R³)ₙ for a compound corresponds in each case to one row of Table A

### Table 135

Compounds of formula I.2* in which R¹ is CH₃, R⁴ is CN, and the combination of NR⁵ and (R³)ₙ for a compound corresponds in each case to one row of Table A

### Table 136

Compounds of formula I.2* in which R¹ is C₂H₅, R⁴ is CN, and the combination of NR⁵ and (R³)ₙ for a compound corresponds in each case to one row of Table A

### Table 137

Compounds of formula I.2* in which R¹ is CH₂-cC₃H₅, R⁴ is CN, and the combination of NR⁵ and (R³)ₙ for a compound corresponds in each case to one row of Table A

### Table 138

Compounds of formula I.2* in which R¹ is CH₂-cC₅H₉, R⁴ is CN, and the combination of NR⁵ and (R³)ₙ for a compound corresponds in each case to one row of Table A

### Table 139

Compounds of formula I.2* in which R¹ is CH₂CH═CH₂, R⁴ is CN, and the combination of NR⁵ and (R³)ₙ for a compound corresponds in each case to one row of Table A

### Table 140

Compounds of formula I.2* in which R¹ is CH₂C≡CH, R⁴ is CN, and the combination of NR⁵ and (R³)ₙ for a compound corresponds in each case to one row of Table A

### Table 141

Compounds of formula I.2* in which R¹ is H, R⁴ is CH₃, and the combination of NR⁵ and (R³)ₙ for a compound corresponds in each case to one row of Table A

### Table 142

Compounds of formula I.2* in which R¹ is CH₃, R⁴ is CH₃, and the combination of NR⁵ and (R³)ₙ for a compound corresponds in each case to one row of Table A

### Table 143

Compounds of formula I.2* in which R¹ is C₂H₅, R⁴ is CH₃, and the combination of NR⁵ and (R³)ₙ for a compound corresponds in each case to one row of Table A

### Table 144

Compounds of formula I.2* in which R¹ is CH₂-cC₃H₅, R⁴ is CH₃, and the combination of NR⁵ and (R³)ₙ for a compound corresponds in each case to one row of Table A

### Table 145

Compounds of formula I.2* in which R¹ is CH₂-cC₅H₉, R⁴ is CH₃, and the combination of NR⁵ and (R³)ₙ for a compound corresponds in each case to one row of Table A

### Table 146

Compounds of formula I.2* in which R¹ is CH₂CH═CH₂, R⁴ is CH₃, and the combination of NR⁵ and (R³)ₙ for a compound corresponds in each case to one row of Table A

### Table 147

Compounds of formula I.2* in which R¹ is CH₂C≡CH, R⁴ is CH₃, and the combination of NR⁵ and (R³)ₙ for a compound corresponds in each case to one row of Table A

### Table 148

Compounds of formula I.2* in which R¹ is H, R⁴ is C₂H₅, and the combination of NR⁵ and (R³)ₙ for a compound corresponds in each case to one row of Table A

### Table 149

Compounds of formula I.2* in which R¹ is CH₃, R⁴ is C₂H₅, and the combination of NR⁵ and (R³)ₙ for a compound corresponds in each case to one row of Table A

### Table 150

Compounds of formula I.2* in which R¹ is C₂H₅, R⁴ is C₂H₅, and the combination of NR⁵ and (R³)ₙ for a compound corresponds in each case to one row of Table A

### Table 151

Compounds of formula I.2* in which R¹ is CH₂-cC₃H₅, R⁴ is C₂H₅, and the combination of NR⁵ and (R³)ₙ for a compound corresponds in each case to one row of Table A

### Table 152

Compounds of formula I.2* in which R¹ is CH₂-cC₅H₉, R⁴ is C₂H₅, and the combination of NR⁵ and (R³)ₙ for a compound corresponds in each case to one row of Table A

### Table 153

Compounds of formula I.2* in which R¹ is CH₂CH═CH₂, R⁴ is C₂H₅, and the combination of NR⁵ and (R³)ₙ for a compound corresponds in each case to one row of Table A

### Table 154

Compounds of formula I.2* in which R¹ is CH₂C≡CH, R⁴ is C₂H₅, and the combination of NR⁵ and (R³)ₙ for a compound corresponds in each case to one row of Table A

### Table 155

Compounds of formula I.2* in which R¹ is H, R⁴ is CH₂CH₂CH₃, and the combination of NR⁵ and (R³)ₙ for a compound corresponds in each case to one row of Table A

### Table 156

Compounds of formula I.2* in which R¹ is CH₃, R⁴ is CH₂CH₂CH₃, and the combination of NR⁵ and (R³)ₙ for a compound corresponds in each case to one row of Table A

### Table 157

Compounds of formula I.2* in which R¹ is C₂H₅, R⁴ is CH₂CH₂CH₃, and the combination of NR⁵ and (R³)ₙ for a compound corresponds in each case to one row of Table A

### Table 158

Compounds of formula I.2* in which R¹ is CH₂-cC₃H₅, R⁴ is CH₂CH₂CH₃, and the combination of X and (R³)ₙ for a compound corresponds in each case to one row of Table A

### Table 159

Compounds of formula I.2* in which R¹ is CH₂-cC₅H₉, R⁴ is CH₂CH₂CH₃, and the combination of NR⁵ and (R³)ₙ for a compound corresponds in each case to one row of Table A

### Table 160

Compounds of formula I.2* in which R¹ is CH₂CH═CH₂, R⁴ is CH₂CH₂CH₃, and the combination of NR⁵ and (R³)ₙ for a compound corresponds in each case to one row of Table A

### Table 161

Compounds of formula I.2* in which R¹ is CH₂C≡CH, R⁴ is CH₂CH₂CH₃, and the combination of NR⁵ and (R³)ₙ for a compound corresponds in each case to one row of Table A

### Table 162

Compounds of formula I.2* in which R¹ is H, R⁴ is CH(CH₃)₂, and the combination of NR⁵ and (R³)ₙ for a compound corresponds in each case to one row of Table A

### Table 163

Compounds of formula I.2* in which R¹ is CH₃, R⁴ is CH(CH₃)₂, and the combination of NR⁵ and (R³)ₙ for a compound corresponds in each case to one row of Table A

### Table 164

Compounds of formula I.2* in which R¹ is C₂H₅, R⁴ is CH(CH₃)₂, and the combination of NR⁵ and (R³)ₙ for a compound corresponds in each case to one row of Table A

### Table 165

Compounds of formula I.2* in which R¹ is CH₂-cC₃H₅, R⁴ is CH(CH₃)₂, and the combination of NR⁵ and (R³)ₙ for a compound corresponds in each case to one row of Table A

### Table 166

Compounds of formula I.2* in which R¹ is CH₂-cC₅H₉, R⁴ is CH(CH₃)₂, and the combination of NR⁵ and (R³)ₙ for a compound corresponds in each case to one row of Table A

### Table 167

Compounds of formula I.2* in which R¹ is CH₂CH═CH₂, R⁴ is CH(CH₃)₂, and the combination of NR⁵ and (R³)ₙ for a compound corresponds in each case to one row of Table A

### Table 168

Compounds of formula I.2* in which R¹ is CH₂C≡CH, R⁴ is CH(CH₃)₂, and the combination of NR⁵ and (R³)ₙ for a compound corresponds in each case to one row of Table A

### Table 169

Compounds of formula I.2* in which R¹ is H, R⁴ is C(═O)NH₂, and the combination of NR⁵ and (R³)ₙ for a compound corresponds in each case to one row of Table A

### Table 170

Compounds of formula I.2* in which R¹ is CH₃, R⁴ is C(═O)NH₂, and the combination of NR⁵ and (R³)ₙ for a compound corresponds in each case to one row of Table A

### Table 171

Compounds of formula I.2* in which R¹ is C₂H₅, R⁴ is C(═O)NH₂, and the combination of NR⁵ and (R³)ₙ for a compound corresponds in each case to one row of Table A

### Table 172

Compounds of formula I.2* in which R¹ is CH₂-cC₃H₅, R⁴ is C(═O)NH₂, and the combination of NR⁵ and (R³)ₙ for a compound corresponds in each case to one row of Table A

### Table 173

Compounds of formula I.2* in which R¹ is CH₂-cC₅H₉, R⁴ is C(═O)NH₂, and the combination of NR⁵ and (R³)ₙ for a compound corresponds in each case to one row of Table A

### Table 174

Compounds of formula I.2* in which R¹ is CH₂CH═CH₂, R⁴ is C(═O)NH₂, and the combination of NR⁵ and (R³)ₙ for a compound corresponds in each case to one row of Table A

### Table 175

Compounds of formula I.2* in which R¹ is CH₂C≡CH, R⁴ is C(═O)NH₂, and the combination of NR⁵ and (R³)ₙ for a compound corresponds in each case to one row of Table A

### Table 176

Compounds of formula I.2* in which R¹ is H, R⁴ is C(═O)NHCH₃, and the combination of NR⁵ and (R³)ₙ for a compound corresponds in each case to one row of Table A

### Table 177

Compounds of formula I.2* in which R¹ is CH₃, R⁴ is C(═O)NHCH₃, and the combination of NR⁵ and (R³)ₙ for a compound corresponds in each case to one row of Table A

### Table 178

Compounds of formula I.2* in which R¹ is C₂H₅, R⁴ is C(═O)NHCH₃, and the combination of NR⁵ and (R³)ₙ for a compound corresponds in each case to one row of Table A

### Table 179

Compounds of formula I.2* in which R¹ is CH₂-cC₃H₅, R⁴ is C(═O)NHCH₃, and the combination of NR⁵ and (R³)ₙ for a compound corresponds in each case to one row of Table A

### Table 180

Compounds of formula I.2* in which R¹ is CH₂-cC₅H₉, R⁴ is C(═O)NHCH₃, and the combination of NR⁵ and (R³)ₙ for a compound corresponds in each case to one row of Table A

### Table 181

Compounds of formula I.2* in which R¹ is CH₂CH═CH₂, R⁴ is C(═O)NHCH₃, and the combination of NR⁵ and (R³)ₙ for a compound corresponds in each case to one row of Table A

### Table 182

Compounds of formula I.2* in which R¹ is CH₂C≡CH, R⁴ is C(═O)NHCH₃, and the combination of NR⁵ and (R³)ₙ for a compound corresponds in each case to one row of Table A

### Table 183

Compounds of formula I.2* in which R¹ is H, R⁴ is C(═O)NHC₂H₅, and the combination of NR⁵ and (R³)ₙ for a compound corresponds in each case to one row of Table A

### Table 184

Compounds of formula I.2* in which R¹ is CH₃, R⁴ is C(═O)NHC₂H₅, and the combination of NR⁵ and (R³)ₙ for a compound corresponds in each case to one row of Table A

### Table 185

Compounds of formula I.2* in which R¹ is C₂H₅, R⁴ is C(═O)NHC₂H₅, and the combination of NR⁵ and (R³)ₙ for a compound corresponds in each case to one row of Table A

### Table 186

Compounds of formula I.2* in which R¹ is CH₂-cC₃H₅, R⁴ is C(═O)NHC₂H₅, and the combination of NR⁵ and (R³)ₙ for a compound corresponds in each case to one row of Table A

### Table 187

Compounds of formula I.2* in which R¹ is CH₂-cC₅H₉, R⁴ is C(═O)NHC₂H₅, and the combination of NR⁵ and (R³)ₙ for a compound corresponds in each case to one row of Table A

### Table 188

Compounds of formula I.2* in which R¹ is CH₂CH═CH₂, R⁴ is C(═O)NHC₂H₅, and the combination of NR⁵ and (R³)ₙ for a compound corresponds in each case to one row of Table A

### Table 189

Compounds of formula I.2* in which R¹ is CH₂C≡CH, R⁴ is C(═O)NHC₂H₅, and the combination of NR⁵ and (R³)ₙ for a compound corresponds in each case to one row of Table A

### Table 190

Compounds of formula I.2* in which R¹ is H, R⁴ is C(═O)N(CH₃)₂, and the combination of NR⁵ and (R³)ₙ for a compound corresponds in each case to one row of Table A

### Table 191

Compounds of formula I.2* in which R¹ is CH₃, R⁴ is C(═O)N(CH₃)₂, and the combination of NR⁵ and (R³)ₙ for a compound corresponds in each case to one row of Table A

### Table 192

Compounds of formula I.2* in which R¹ is C₂H₅, R⁴ is C(═O)N(CH₃)₂, and the combination of NR⁵ and (R³)ₙ for a compound corresponds in each case to one row of Table A

### Table 193

Compounds of formula I.2* in which R¹ is CH₂-cC₃H₅, R⁴ is C(═O)N(CH₃)₂, and the combination of NR⁵ and (R³)ₙ for a compound corresponds in each case to one row of Table A

### Table 194

Compounds of formula I.2* in which R¹ is CH₂-cC₅H₉, R⁴ is C(═O)N(CH₃)₂, and the combination of NR⁵ and (R³)ₙ for a compound corresponds in each case to one row of Table A

### Table 195

Compounds of formula I.2* in which R¹ is CH₂CH═CH₂, R⁴ is C(═O)N(CH₃)₂, and the combination of NR⁵ and (R³)ₙ for a compound corresponds in each case to one row of Table A

### Table 196

Compounds of formula I.2* in which R¹ is CH₂C≡CH, R⁴ is C(═O)N(CH₃)₂, and the combination of NR⁵ and (R³)ₙ for a compound corresponds in each case to one row of Table A

### Table 197

Compounds of formula I.2* in which R¹ is H, R⁴ is C(═O)N(CH₃)C₂H₅, and the combination of NR⁵ and (R³)ₙ for a compound corresponds in each case to one row of Table A

### Table 198

Compounds of formula I.2* in which R¹ is CH₃, R⁴ is C(═O)N(CH₃)C₂H₅, and the combination of NR⁵ and (R³)ₙ for a compound corresponds in each case to one row of Table A

### Table 199

Compounds of formula I.2* in which R¹ is C₂H₅, R⁴ is C(═O)N(CH₃)C₂H₅, and the combination of NR⁵ and (R³)ₙ for a compound corresponds in each case to one row of Table A

### Table 200

Compounds of formula I.2* in which R¹ is CH₂-cC₃H₅, R⁴ is C(═O)N(CH₃)C₂H₅, and the combination of NR⁵ and (R³)ₙ for a compound corresponds in each case to one row of Table A

### Table 201

Compounds of formula I.2* in which R¹ is CH₂-cC₅H₉, R⁴ is C(═O)N(CH₃)C₂H₅, and the combination of NR⁵ and (R³)ₙ for a compound corresponds in each case to one row of Table A

### Table 202

Compounds of formula I.2* in which R¹ is CH₂CH═CH₂, R⁴ is C(═O)N(CH₃)C₂H₅, and the combination of NR⁵ and (R³)ₙ for a compound corresponds in each case to one row of Table A

### Table 203

Compounds of formula I.2* in which R¹ is CH₂C≡CH, R⁴ is C(═O)N(CH₃)C₂H₅, and the combination of NR⁵ and (R³)ₙ for a compound corresponds in each case to one row of Table A

### Table 204

Compounds of formula I.2* in which R¹ is H, R⁴ is C(═O)OCH₃, and the combination of NR⁵ and (R³)ₙ for a compound corresponds in each case to one row of Table A

### Table 205

Compounds of formula I.2* in which R¹ is CH₃, R⁴ is C(═O)OCH₃, and the combination of NR⁵ and (R³)ₙ for a compound corresponds in each case to one row of Table A

### Table 206

Compounds of formula I.2* in which R¹ is C₂H₅, R⁴ is C(═O)OCH₃, and the combination of NR⁵ and (R³)ₙ for a compound corresponds in each case to one row of Table A

### Table 207

Compounds of formula I.2* in which R¹ is CH₂-cC₃H₅, R⁴ is C(═O)OCH₃, and the combination of NR⁵ and (R³)ₙ for a compound corresponds in each case to one row of Table A

### Table 208

Compounds of formula I.2* in which R¹ is CH₂-cC₅H₉, R⁴ is C(═O)OCH₃, and the combination of NR⁵ and (R³)ₙ for a compound corresponds in each case to one row of Table A

### Table 209

Compounds of formula I.2* in which R¹ is CH₂CH═CH₂, R⁴ is C(═O)OCH₃, and the combination of NR⁵ and (R³)ₙ for a compound corresponds in each case to one row of Table A

### Table 210

Compounds of formula I.2* in which R¹ is CH₂C≡CH, R⁴ is C(═O)OCH₃, and the combination of NR⁵ and (R³)ₙ for a compound corresponds in each case to one row of Table A

### Table 211

Compounds of formula I.2* in which R¹ is H, R⁴ is C(═O)OC₂H₅, and the combination of NR⁵ and (R³)ₙ for a compound corresponds in each case to one row of Table A

### Table 212

Compounds of formula I.2* in which R¹ is CH₃, R⁴ is C(═O)OC₂H₅, and the combination of NR⁵ and (R³)ₙ for a compound corresponds in each case to one row of Table A

### Table 213

Compounds of formula I.2* in which R¹ is C₂H₅, R⁴ is C(═O)OC₂H₅, and the combination of NR⁵ and (R³)ₙ for a compound corresponds in each case to one row of Table A

### Table 214

Compounds of formula I.2* in which R¹ is CH₂-cC₃H₅, R⁴ is C(═O)OC₂H₅, and the combination of NR⁵ and (R³)ₙ for a compound corresponds in each case to one row of Table A

### Table 215

Compounds of formula I.2* in which R¹ is CH₂-cC₅H₉, R⁴ is C(═O)OC₂H₅, and the combination of NR⁵ and (R³)ₙ for a compound corresponds in each case to one row of Table A

### Table 216

Compounds of formula I.2* in which R¹ is CH₂CH═CH₂, R⁴ is C(═O)OC₂H₅, and the combination of NR⁵ and (R³)ₙ for a compound corresponds in each case to one row of Table A

### Table 217

Compounds of formula I.2* in which R¹ is CH₂C≡CH, R⁴ is C(═O)OC₂H₅, and the combination of NR⁵ and (R³)ₙ for a compound corresponds in each case to one row of Table A

### Table 218

Compounds of formula I.2* in which R¹ is H, R⁴ is NH₂, and the combination of NR⁵ and (R³)ₙ for a compound corresponds in each case to one row of Table A

### Table 219

Compounds of formula I.2* in which R¹ is CH₃, R⁴ is NH₂, and the combination of NR⁵ and (R³)ₙ for a compound corresponds in each case to one row of Table A

### Table 220

Compounds of formula I.2* in which R¹ is C₂H₅, R⁴ is NH₂, and the combination of NR⁵ and (R³)ₙ for a compound corresponds in each case to one row of Table A

### Table 221

Compounds of formula I.2* in which R¹ is CH₂-cC₃H₅, R⁴ is NH₂, and the combination of NR⁵ and (R³)ₙ for a compound corresponds in each case to one row of Table A

### Table 222

Compounds of formula I.2* in which R¹ is CH₂-cC₅H₉, R⁴ is NH₂, and the combination of NR⁵ and (R³)ₙ for a compound corresponds in each case to one row of Table A

### Table 223

Compounds of formula I.2* in which R¹ is CH₂CH═CH₂, R⁴ is NH₂, and the combination of NR⁵ and (R³)ₙ for a compound corresponds in each case to one row of Table A

### Table 224

Compounds of formula I.2* in which R¹ is CH₂C≡CH, R⁴ is NH₂, and the combination of NR⁵ and (R³)ₙ for a compound corresponds in each case to one row of Table A

### Table 225

Compounds of formula I.2* in which R¹ is H, R⁴ is NHCH₃, and the combination of NR⁵ and (R³)ₙ for a compound corresponds in each case to one row of Table A

### Table 226

Compounds of formula I.2* in which R¹ is CH₃, R⁴ is NHCH₃, and the combination of NR⁵ and (R³)ₙ for a compound corresponds in each case to one row of Table A

### Table 227

Compounds of formula I.2* in which R¹ is C₂H₅, R⁴ is NHCH₃, and the combination of NR⁵ and (R³)ₙ for a compound corresponds in each case to one row of Table A

### Table 228

Compounds of formula I.2* in which R¹ is CH₂-cC₃H₅, R⁴ is NHCH₃, and the combination of NR⁵ and (R³)ₙ for a compound corresponds in each case to one row of Table A

### Table 229

Compounds of formula I.2* in which R¹ is CH₂-cC₅H₉, R⁴ is NHCH₃, and the combination of NR⁵ and (R³)ₙ for a compound corresponds in each case to one row of Table A

### Table 230

Compounds of formula I.2* in which R¹ is CH₂CH═CH₂, R⁴ is NHCH₃, and the combination of NR⁵ and (R³)ₙ for a compound corresponds in each case to one row of Table A

### Table 231

Compounds of formula I.2* in which R¹ is CH₂C≡CH, R⁴ is NHCH₃, and the combination of NR⁵ and (R³)ₙ for a compound corresponds in each case to one row of Table A

### Table 232

Compounds of formula I.2* in which R¹ is H, R⁴ is N(CH₃)C₂H₅, and the combination of NR⁵ and (R³)ₙ for a compound corresponds in each case to one row of Table A

### Table 233

Compounds of formula I.2* in which R¹ is CH₃, R⁴ is N(CH₃)C₂H₅, and the combination of NR⁵ and (R³)ₙ for a compound corresponds in each case to one row of Table A

### Table 234

Compounds of formula I.2* in which R¹ is C₂H₅, R⁴ is N(CH₃)C₂H₅, and the combination of NR⁵ and (R³)ₙ for a compound corresponds in each case to one row of Table A

### Table 235

Compounds of formula I.2* in which R¹ is CH₂-cC₃H₅, R⁴ is N(CH₃)C₂H₅, and the combination of NR⁵ and (R³)ₙ for a compound corresponds in each case to one row of Table A

### Table 236

Compounds of formula I.2* in which R¹ is CH₂-cC₅H₉, R⁴ is N(CH₃)C₂H₅, and the combination of NR⁵ and (R³)ₙ for a compound corresponds in each case to one row of Table A

### Table 237

Compounds of formula I.2* in which R¹ is CH₂CH═CH₂, R⁴ is N(CH₃)C₂H₅, and the combination of NR⁵ and (R³)ₙ for a compound corresponds in each case to one row of Table A

### Table 238

Compounds of formula I.2* in which R¹ is CH₂C≡CH, R⁴ is N(CH₃)C₂H₅, and the combination of NR⁵ and (R³)ₙ for a compound corresponds in each case to one row of Table A

### Table 239

Compounds of formula I.2* in which R¹ is H, R⁴ is NHCH₂CN, and the combination of NR⁵ and (R³)ₙ for a compound corresponds in each case to one row of Table A

### Table 240

Compounds of formula I.2* in which R¹ is CH₃, R⁴ is NHCH₂CN, and the combination of NR⁵ and (R³)ₙ for a compound corresponds in each case to one row of Table A

### Table 241

Compounds of formula I.2* in which R¹ is C₂H₅, R⁴ is NHCH₂CN, and the combination of NR⁵ and (R³)ₙ for a compound corresponds in each case to one row of Table A

### Table 242

Compounds of formula I.2* in which R¹ is CH₂-cC₃H₅, R⁴ is NHCH₂CN, and the combination of NR⁵ and (R³)ₙ for a compound corresponds in each case to one row of Table A

### Table 243

Compounds of formula I.1* in which R¹ is CH₂-cC₅H₉, R⁴ is NHCH₂CN, and the combination of NR⁵ and (R³)ₙ for a compound corresponds in each case to one row of Table A

### Table 244

Compounds of formula I.2* in which R¹ is CH₂CH═CH₂, R⁴ is NHCH₂CN, and the combination of NR⁵ and (R³)ₙ for a compound corresponds in each case to one row of Table A

### Table 245

Compounds of formula I.2* in which R¹ is CH₂C≡CH, R⁴ is NHCH₂CN, and the combination of NR⁵ and (R³)ₙ for a compound corresponds in each case to one row of Table A

### Table 246

Compounds of formula I.2* in which R¹ is H, R⁴ is 4,5-dihydrooxazol-2-yl, and the combination of NR⁵ and (R³)ₙ for a compound corresponds in each case to one row of Table A

### Table 247

Compounds of formula I.2* in which R¹ is CH₃, R⁴ is 4,5-dihydrooxazol-2-yl, and the combination of NR⁵ and (R³)ₙ for a compound corresponds in each case to one row of Table A

### Table 248

Compounds of formula I.2* in which R¹ is C₂H₅, R⁴ is 4,5-dihydrooxazol-2-yl, and the combination of NR⁵ and (R³)ₙ for a compound corresponds in each case to one row of Table A

### Table 249

Compounds of formula I.2* in which R¹ is CH₂-cC₃H₅, R⁴ is 4,5-dihydrooxazol-2-yl, and the combination of NR⁵ and (R³)ₙ for a compound corresponds in each case to one row of Table A

### Table 250

Compounds of formula I.2* in which R¹ is CH₂-cC₅H₉, R⁴ is 4,5-dihydrooxazol-2-yl, and the combination of NR⁵ and (R³)ₙ for a compound corresponds in each case to one row of Table A

### Table 251

Compounds of formula I.2* in which R¹ is CH₂CH═CH₂, R⁴ is 4,5-dihydrooxazol-2-yl, and the combination of NR⁵ and (R³)ₙ for a compound corresponds in each case to one row of Table A

### Table 252

Compounds of formula I.2* in which R¹ is CH₂C≡CH, R⁴ is 4,5-dihydrooxazol-2-yl, and the combination of NR⁵ and (R³)ₙ for a compound corresponds in each case to one row of Table A

TABLE A

| No. | NR⁵ | (R³)ₙ |
|---|---|---|
| A-1 | NOH | 3,5-F₂ |
| A-2 | (Z)-N-OCH₃ | 3,5-F₂ |
| A-3 | (E)-N-OCH₃ | 3,5-F₂ |
| A-4 | (Z)-N-OC₂H₅ | 3,5-F₂ |
| A-5 | (E)-N-OC₂H₅ | 3,5-F₂ |
| A-6 | (Z)-N-OCH₂CF₃ | 3,5-F₂ |
| A-7 | (E)-N-OCH₂CF₃ | 3,5-F₂ |
| A-8 | (Z)-N-OCH₂C₆H₅ | 3,5-F₂ |
| A-9 | (E)-N-OCH₂C₆H₅ | 3,5-F₂ |
| A-10 | NOH | 3,5-Cl₂ |
| A-11 | (Z)-N-OCH₃ | 3,5-Cl₂ |
| A-12 | (E)-N-OCH₃ | 3,5-Cl₂ |
| A-13 | (Z)-N-OC₂H₅ | 3,5-Cl₂ |
| A-14 | (E)-N-OC₂H₅ | 3,5-Cl₂ |
| A-15 | (Z)-N-OCH₂CF₃ | 3,5-Cl₂ |
| A-16 | (E)-N-OCH₂CF₃ | 3,5-Cl₂ |
| A-17 | (Z)-N-OCH₂C₆H₅ | 3,5-Cl₂ |
| A-18 | (E)-N-OCH₂C₆H₅ | 3,5-Cl₂ |
| A-19 | NOH | 3,5-Br₂ |
| A-20 | (Z)-N-OCH₃ | 3,5-Br₂ |
| A-21 | (E)-N-OCH₃ | 3,5-Br₂ |
| A-22 | (Z)-N-OC₂H₅ | 3,5-Br₂ |
| A-23 | (E)-N-OC₂H₅ | 3,5-Br₂ |
| A-24 | (Z)-N-OCH₂CF₃ | 3,5-Br₂ |
| A-25 | (E)-N-OCH₂CF₃ | 3,5-Br₂ |
| A-26 | (Z)-N-OCH₂C₆H₅ | 3,5-Br₂ |
| A-27 | (E)-N-OCH₂C₆H₅ | 3,5-Br₂ |
| A-28 | NOH | 3,5-I₂ |
| A-29 | (Z)-N-OCH₃ | 3,5-I₂ |
| A-30 | (E)-N-OCH₃ | 3,5-I₂ |
| A-31 | (Z)-N-OC₂H₅ | 3,5-I₂ |
| A-32 | (E)-N-OC₂H₅ | 3,5-I₂ |
| A-33 | (Z)-N-OCH₂CF₃ | 3,5-I₂ |
| A-34 | (E)-N-OCH₂CF₃ | 3,5-I₂ |
| A-35 | (Z)-N-OCH₂C₆H₅ | 3,5-I₂ |
| A-36 | (E)-N-OCH₂C₆H₅ | 3,5-I₂ |
| A-37 | NOH | 3,5-(CF₃)₂ |
| A-38 | (Z)-N-OCH₃ | 3,5-(CF₃)₂ |
| A-39 | (E)-N-OCH₃ | 3,5-(CF₃)₂ |
| A-40 | (Z)-N-OC₂H₅ | 3,5-(CF₃)₂ |
| A-41 | (E)-N-OC₂H₅ | 3,5-(CF₃)₂ |
| A-42 | (Z)-N-OCH₂CF₃ | 3,5-(CF₃)₂ |
| A-43 | (E)-N-OCH₂CF₃ | 3,5-(CF₃)₂ |
| A-44 | (Z)-N-OCH₂C₆H₅ | 3,5-(CF₃)₂ |
| A-45 | (E)-N-OCH₂C₆H₅ | 3,5-(CF₃)₂ |
| A-46 | NOH | 3-Cl,5-F |
| A-47 | (Z)-N-OCH₃ | 3-Cl,5-F |
| A-48 | (E)-N-OCH₃ | 3-Cl,5-F |
| A-49 | (Z)-N-OC₂H₅ | 3-Cl,5-F |
| A-50 | (E)-N-OC₂H₅ | 3-Cl,5-F |
| A-51 | (Z)-N-OCH₂CF₃ | 3-Cl,5-F |
| A-52 | (E)-N-OCH₂CF₃ | 3-Cl,5-F |
| A-53 | (Z)-N-OCH₂C₆H₅ | 3-Cl,5-F |
| A-54 | (E)-N-OCH₂C₆H₅ | 3-Cl,5-F |
| A-55 | NOH | 3-Cl,5-Br |
| A-56 | (Z)-N-OCH₃ | 3-Cl,5-Br |
| A-57 | (E)-N-OCH₃ | 3-Cl,5-Br |
| A-58 | (Z)-N-OC₂H₅ | 3-Cl,5-Br |
| A-59 | (E)-N-OC₂H₅ | 3-Cl,5-Br |
| A-60 | (Z)-N-OCH₂CF₃ | 3-Cl,5-Br |
| A-61 | (E)-N-OCH₂CF₃ | 3-Cl,5-Br |
| A-62 | (Z)-N-OCH₂C₆H₅ | 3-Cl,5-Br |
| A-63 | (E)-N-OCH₂C₆H₅ | 3-Cl,5-Br |
| A-64 | NOH | 3-Cl,5-I |
| A-65 | (Z)-N-OCH₃ | 3-Cl,5-I |
| A-66 | (E)-N-OCH₃ | 3-Cl,5-I |
| A-67 | (Z)-N-OC₂H₅ | 3-Cl,5-I |
| A-68 | (E)-N-OC₂H₅ | 3-Cl,5-I |
| A-69 | (Z)-N-OCH₂CF₃ | 3-Cl,5-I |
| A-70 | (E)-N-OCH₂CF₃ | 3-Cl,5-I |
| A-71 | (Z)-N-OCH₂C₆H₅ | 3-Cl,5-I |
| A-72 | (E)-N-OCH₂C₆H₅ | 3-Cl,5-I |
| A-73 | NOH | 3-F,5-CN |
| A-74 | (Z)-N-OCH₃ | 3-F,5-CN |
| A-75 | (E)-N-OCH₃ | 3-F,5-CN |
| A-76 | (Z)-N-OC₂H₅ | 3-F,5-CN |
| A-77 | (E)-N-OC₂H₅ | 3-F,5-CN |
| A-78 | (Z)-N-OCH₂CF₃ | 3-F,5-CN |
| A-79 | (E)-N-OCH₂CF₃ | 3-F,5-CN |
| A-80 | (Z)-N-OCH₂C₆H₅ | 3-F,5-CN |
| A-81 | (E)-N-OCH₂C₆H₅ | 3-F,5-CN |
| A-82 | NOH | 3-Cl,5-CN |
| A-83 | (Z)-N-OCH₃ | 3-Cl,5-CN |
| A-84 | (E)-N-OCH₃ | 3-Cl,5-CN |
| A-85 | (Z)-N-OC₂H₅ | 3-Cl,5-CN |
| A-86 | (E)-N-OC₂H₅ | 3-Cl,5-CN |
| A-87 | (Z)-N-OCH₂CF₃ | 3-Cl,5-CN |
| A-88 | (E)-N-OCH₂CF₃ | 3-Cl,5-CN |
| A-89 | (Z)-N-OCH₂C₆H₅ | 3-Cl,5-CN |
| A-90 | (E)-N-OCH₂C₆H₅ | 3-Cl,5-CN |
| A-91 | NOH | 3-CF₃,5-CN |
| A-92 | (Z)-N-OCH₃ | 3-CF₃,5-CN |
| A-93 | (E)-N-OCH₃ | 3-CF₃,5-CN |
| A-94 | (Z)-N-OC₂H₅ | 3-CF₃,5-CN |
| A-95 | (E)-N-OC₂H₅ | 3-CF₃,5-CN |
| A-96 | (Z)-N-OCH₂CF₃ | 3-CF₃,5-CN |
| A-97 | (E)-N-OCH₂CF₃ | 3-CF₃,5-CN |
| A-98 | (Z)-N-OCH₂C₆H₅ | 3-CF₃,5-CN |
| A-99 | (E)-N-OCH₂C₆H₅ | 3-CF₃,5-CN |
| A-100 | NOH | 3-F,5-CF₃ |
| A-101 | (Z)-N-OCH₃ | 3-F,5-CF₃ |
| A-102 | (E)-N-OCH₃ | 3-F,5-CF₃ |
| A-103 | (Z)-N-OC₂H₅ | 3-F,5-CF₃ |
| A-104 | (E)-N-OC₂H₅ | 3-F,5-CF₃ |
| A-105 | (Z)-N-OCH₂CF₃ | 3-F,5-CF₃ |
| A-106 | (E)-N-OCH₂CF₃ | 3-F,5-CF₃ |
| A-107 | (Z)-N-OCH₂C₆H₅ | 3-F,5-CF₃ |
| A-108 | (E)-N-OCH₂C₆H₅ | 3-F,5-CF₃ |
| A-109 | NOH | 3-Cl,5-CF₃ |
| A-110 | (Z)-N-OCH₃ | 3-Cl,5-CF₃ |
| A-111 | (E)-N-OCH₃ | 3-Cl,5-CF₃ |
| A-112 | (Z)-N-OC₂H₅ | 3-Cl,5-CF₃ |
| A-113 | (E)-N-OC₂H₅ | 3-Cl,5-CF₃ |
| A-114 | (Z)-N-OCH₂CF₃ | 3-Cl,5-CF₃ |
| A-115 | (E)-N-OCH₂CF₃ | 3-Cl,5-CF₃ |
| A-116 | (Z)-N-OCH₂C₆H₅ | 3-Cl,5-CF₃ |
| A-117 | (E)-N-OCH₂C₆H₅ | 3-Cl,5-CF₃ |
| A-118 | NOH | 3-Br,5-CF₃ |
| A-119 | (Z)-N-OCH₃ | 3-Br,5-CF₃ |
| A-120 | (E)-N-OCH₃ | 3-Br,5-CF₃ |
| A-121 | (Z)-N-OC₂H₅ | 3-Br,5-CF₃ |
| A-122 | (E)-N-OC₂H₅ | 3-Br,5-CF₃ |
| A-123 | (Z)-N-OCH₂CF₃ | 3-Br,5-CF₃ |
| A-124 | (E)-N-OCH₂CF₃ | 3-Br,5-CF₃ |
| A-125 | (Z)-N-OCH₂C₆H₅ | 3-Br,5-CF₃ |
| A-126 | (E)-N-OCH₂C₆H₅ | 3-Br,5-CF₃ |
| A-127 | NOH | 3-I,5-CF₃ |
| A-128 | (Z)-N-OCH₃ | 3-I,5-CF₃ |
| A-129 | (E)-N-OCH₃ | 3-I,5-CF₃ |
| A-130 | (Z)-N-OC₂H₅ | 3-I,5-CF₃ |
| A-131 | (E)-N-OC₂H₅ | 3-I,5-CF₃ |
| A-132 | (Z)-N-OCH₂CF₃ | 3-I,5-CF₃ |
| A-133 | (E)-N-OCH₂CF₃ | 3-I,5-CF₃ |
| A-134 | (Z)-N-OCH₂C₆H₅ | 3-I,5-CF₃ |
| A-135 | (E)-N-OCH₂C₆H₅ | 3-I,5-CF₃ |
| A-136 | NOH | 3-Cl,5-SO₂CH₃ |
| A-137 | (Z)-N-OCH₃ | 3-Cl,5-SO₂CH₃ |
| A-138 | (E)-N-OCH₃ | 3-Cl,5-SO₂CH₃ |
| A-139 | (Z)-N-OC₂H₅ | 3-Cl,5-SO₂CH₃ |
| A-140 | (E)-N-OC₂H₅ | 3-Cl,5-SO₂CH₃ |
| A-141 | (Z)-N-OCH₂CF₃ | 3-Cl,5-SO₂CH₃ |
| A-142 | (E)-N-OCH₂CF₃ | 3-Cl,5-SO₂CH₃ |
| A-143 | (Z)-N-OCH₂C₆H₅ | 3-Cl,5-SO₂CH₃ |
| A-144 | (E)-N-OCH₂C₆H₅ | 3-Cl,5-SO₂CH₃ |
| A-145 | NOH | 3-Cl,5-SO₂CF₃ |
| A-146 | (Z)-N-OCH₃ | 3-Cl,5-SO₂CF₃ |
| A-147 | (E)-N-OCH₃ | 3-Cl,5-SO₂CF₃ |
| A-148 | (Z)-N-OC₂H₅ | 3-Cl,5-SO₂CF₃ |
| A-149 | (E)-N-OC₂H₅ | 3-Cl,5-SO₂CF₃ |
| A-150 | (Z)-N-OCH₂CF₃ | 3-Cl,5-SO₂CF₃ |
| A-151 | (E)-N-OCH₂CF₃ | 3-Cl,5-SO₂CF₃ |
| A-152 | (Z)-N-OCH₂C₆H₅ | 3-Cl,5-SO₂CF₃ |
| A-153 | (E)-N-OCH₂C₆H₅ | 3-Cl,5-SO₂CF₃ |
| A-154 | NOH | 3-Cl,5-cC₃H₅ |
| A-155 | (Z)-N-OCH₃ | 3-Cl,5-cC₃H₅ |
| A-156 | (E)-N-OCH₃ | 3-Cl,5-cC₃H₅ |
| A-157 | (Z)-N-OC₂H₅ | 3-Cl,5-cC₃H₅ |
| A-158 | (E)-N-OC₂H₅ | 3-Cl,5-cC₃H₅ |
| A-159 | (Z)-N-OCH₂CF₃ | 3-Cl,5-cC₃H₅ |
| A-160 | (E)-N-OCH₂CF₃ | 3-Cl,5-cC₃H₅ |
| A-161 | (Z)-N-OCH₂C₆H₅ | 3-Cl,5-cC₃H₅ |
| A-162 | (E)-N-OCH₂C₆H₅ | 3-Cl,5-cC₃H₅ |
| A-163 | NOH | 3-Cl,5-[2,2-Cl₂-cC₃H₃] |
| A-164 | (Z)-N-OCH₃ | 3-Cl,5-[2,2-Cl₂-cC₃H₃] |
| A-165 | (E)-N-OCH₃ | 3-Cl,5-[2,2-Cl₂-cC₃H₃] |
| A-166 | (Z)-N-OC₂H₅ | 3-Cl,5-[2,2-Cl₂-cC₃H₃] |
| A-167 | (E)-N-OC₂H₅ | 3-Cl,5-[2,2-Cl₂-cC₃H₃] |
| A-168 | (Z)-N-OCH₂CF₃ | 3-Cl,5-[2,2-Cl₂-cC₃H₃] |
| A-169 | (E)-N-OCH₂CF₃ | 3-Cl,5-[2,2-Cl₂-cC₃H₃] |
| A-170 | (Z)-N-OCH₂C₆H₅ | 3-Cl,5-[2,2-Cl₂-cC₃H₃] |
| A-171 | (E)-N-OCH₂C₆H₅ | 3-Cl,5-[2,2-Cl₂-cC₃H₃] |
| A-172 | NOH | 3-Cl,5-OCF₃ |
| A-173 | (Z)-N-OCH₃ | 3-Cl,5-OCF₃ |
| A-174 | (E)-N-OCH₃ | 3-Cl,5-OCF₃ |
| A-175 | (Z)-N-OC₂H₅ | 3-Cl,5-OCF₃ |
| A-176 | (E)-N-OC₂H₅ | 3-Cl,5-OCF₃ |
| A-177 | (Z)-N-OCH₂CF₃ | 3-Cl,5-OCF₃ |
| A-178 | (E)-N-OCH₂CF₃ | 3-Cl,5-OCF₃ |
| A-179 | (Z)-N-OCH₂C₆H₅ | 3-Cl,5-OCF₃ |
| A-180 | (E)-N-OCH₂C₆H₅ | 3-Cl,5-OCF₃ |
| A-181 | NOH | 3-Br,5-OCF₃ |
| A-182 | (Z)-N-OCH₃ | 3-Br,5-OCF₃ |
| A-183 | (E)-N-OCH₃ | 3-Br,5-OCF₃ |
| A-184 | (Z)-N-OC₂H₅ | 3-Br,5-OCF₃ |
| A-185 | (E)-N-OC₂H₅ | 3-Br,5-OCF₃ |
| A-186 | (Z)-N-OCH₂CF₃ | 3-Br,5-OCF₃ |
| A-187 | (E)-N-OCH₂CF₃ | 3-Br,5-OCF₃ |
| A-188 | (Z)-N-OCH₂C₆H₅ | 3-Br,5-OCF₃ |
| A-189 | (E)-N-OCH₂C₆H₅ | 3-Br,5-OCF₃ |
| A-190 | NOH | 3-F,5-cC₃H₅ |
| A-191 | (Z)-N-OCH₃ | 3-F,5-cC₃H₅ |
| A-192 | (E)-N-OCH₃ | 3-F,5-cC₃H₅ |
| A-193 | (Z)-N-OC₂H₅ | 3-F,5-cC₃H₅ |
| A-194 | (E)-N-OC₂H₅ | 3-F,5-cC₃H₅ |
| A-195 | (Z)-N-OCH₂CF₃ | 3-F,5-cC₃H₅ |
| A-196 | (E)-N-OCH₂CF₃ | 3-F,5-cC₃H₅ |
| A-197 | (Z)-N-OCH₂C₆H₅ | 3-F,5-cC₃H₅ |
| A-198 | (E)-N-OCH₂C₆H₅ | 3-F,5-cC₃H₅ |
| A-199 | NOH | 3-Cl,5-CH₂CN |
| A-200 | (Z)-N-OCH₃ | 3-Cl,5-CH₂CN |
| A-201 | (E)-N-OCH₃ | 3-Cl,5-CH₂CN |
| A-202 | (Z)-N-OC₂H₅ | 3-Cl,5-CH₂CN |
| A-203 | (E)-N-OC₂H₅ | 3-Cl,5-CH₂CN |
| A-204 | (Z)-N-OCH₂CF₃ | 3-Cl,5-CH₂CN |
| A-205 | (E)-N-OCH₂CF₃ | 3-Cl,5-CH₂CN |
| A-206 | (Z)-N-OCH₂C₆H₅ | 3-Cl,5-CH₂CN |
| A-207 | (E)-N-OCH₂C₆H₅ | 3-Cl,5-CH₂CN |
| A-208 | NOH | 3-Cl,5-C(CH₃)₂CN |
| A-209 | (Z)-N-OCH₃ | 3-Cl,5-C(CH₃)₂CN |
| A-210 | (E)-N-OCH₃ | 3-Cl,5-C(CH₃)₂CN |
| A-211 | (Z)-N-OC₂H₅ | 3-Cl,5-C(CH₃)₂CN |
| A-212 | (E)-N-OC₂H₅ | 3-Cl,5-C(CH₃)₂CN |
| A-213 | (Z)-N-OCH₂CF₃ | 3-Cl,5-C(CH₃)₂CN |
| A-214 | (E)-N-OCH₂CF₃ | 3-Cl,5-C(CH₃)₂CN |
| A-215 | (Z)-N-OCH₂C₆H₅ | 3-Cl,5-C(CH₃)₂CN |
| A-216 | (E)-N-OCH₂C₆H₅ | 3-Cl,5-C(CH₃)₂CN |
| A-217 | NOH | 3-CF₃,5-CH₂CN |
| A-218 | (Z)-N-OCH₃ | 3-CF₃,5-CH₂CN |
| A-219 | (E)-N-OCH₃ | 3-CF₃,5-CH₂CN |
| A-220 | (Z)-N-OC₂H₅ | 3-CF₃,5-CH₂CN |
| A-221 | (E)-N-OC₂H₅ | 3-CF₃,5-CH₂CN |
| A-222 | (Z)-N-OCH₂CF₃ | 3-CF₃,5-CH₂CN |
| A-223 | (E)-N-OCH₂CF₃ | 3-CF₃,5-CH₂CN |
| A-224 | (Z)-N-OCH₂C₆H₅ | 3-CF₃,5-CH₂CN |
| A-225 | (E)-N-OCH₂C₆H₅ | 3-CF₃,5-CH₂CN |
| A-226 | NOH | 3-CF₃,5-C(CH₃)₂CN |
| A-227 | (Z)-N-OCH₃ | 3-CF₃,5-C(CH₃)₂CN |
| A-228 | (E)-N-OCH₃ | 3-CF₃,5-C(CH₃)₂CN |
| A-229 | (Z)-N-OC₂H₅ | 3-CF₃,5-C(CH₃)₂CN |
| A-230 | (E)-N-OC₂H₅ | 3-CF₃,5-C(CH₃)₂CN |
| A-231 | (Z)-N-OCH₂CF₃ | 3-CF₃,5-C(CH₃)₂CN |
| A-232 | (E)-N-OCH₂CF₃ | 3-CF₃,5-C(CH₃)₂CN |
| A-233 | (Z)-N-OCH₂C₆H₅ | 3-CF₃,5-C(CH₃)₂CN |
| A-234 | (E)-N-OCH₂C₆H₅ | 3-CF₃,5-C(CH₃)₂CN |
| A-235 | NOH | 3-CF₃,5-SO₂CH₃ |
| A-236 | (Z)-N-OCH₃ | 3-CF₃,5-SO₂CH₃ |
| A-237 | (E)-N-OCH₃ | 3-CF₃,5-SO₂CH₃ |
| A-238 | (Z)-N-OC₂H₅ | 3-CF₃,5-SO₂CH₃ |
| A-239 | (E)-N-OC₂H₅ | 3-CF₃,5-SO₂CH₃ |
| A-240 | (Z)-N-OCH₂CF₃ | 3-CF₃,5-SO₂CH₃ |
| A-241 | (E)-N-OCH₂CF₃ | 3-CF₃,5-SO₂CH₃ |
| A-242 | (Z)-N-OCH₂C₆H₅ | 3-CF₃,5-SO₂CH₃ |
| A-243 | (E)-N-OCH₂C₆H₅ | 3-CF₃,5-SO₂CH₃ |
| A-244 | NOH | 3-CF₃,5-SO₂CF₃ |
| A-245 | (Z)-N-OCH₃ | 3-CF₃,5-SO₂CF₃ |
| A-246 | (E)-N-OCH₃ | 3-CF₃,5-SO₂CF₃ |
| A-247 | (Z)-N-OC₂H₅ | 3-CF₃,5-SO₂CF₃ |
| A-248 | (E)-N-OC₂H₅ | 3-CF₃,5-SO₂CF₃ |
| A-249 | (Z)-N-OCH₂CF₃ | 3-CF₃,5-SO₂CF₃ |
| A-250 | (E)-N-OCH₂CF₃ | 3-CF₃,5-SO₂CF₃ |
| A-251 | (Z)-N-OCH₂C₆H₅ | 3-CF₃,5-SO₂CF₃ |
| A-252 | (E)-N-OCH₂C₆H₅ | 3-CF₃,5-SO₂CF₃ |
| A-253 | NOH | 3-CF₃,5-OCF₃ |
| A-254 | (Z)-N-OCH₃ | 3-CF₃,5-OCF₃ |
| A-255 | (E)-N-OCH₃ | 3-CF₃,5-OCF₃ |
| A-256 | (Z)-N-OC₂H₅ | 3-CF₃,5-OCF₃ |
| A-257 | (E)-N-OC₂H₅ | 3-CF₃,5-OCF₃ |
| A-258 | (Z)-N-OCH₂CF₃ | 3-CF₃,5-OCF₃ |
| A-259 | (E)-N-OCH₂CF₃ | 3-CF₃,5-OCF₃ |
| A-260 | (Z)-N-OCH₂C₆H₅ | 3-CF₃,5-OCF₃ |
| A-261 | (E)-N-OCH₂C₆H₅ | 3-CF₃,5-OCF₃ |
| A-262 | NOH | 3-CF₃,5-cC₃H₅ |
| A-263 | (Z)-N-OCH₃ | 3-CF₃,5-cC₃H₅ |
| A-264 | (E)-N-OCH₃ | 3-CF₃,5-cC₃H₅ |
| A-265 | (Z)-N-OC₂H₅ | 3-CF₃,5-cC₃H₅ |
| A-266 | (E)-N-OC₂H₅ | 3-CF₃,5-cC₃H₅ |
| A-267 | (Z)-N-OCH₂CF₃ | 3-CF₃,5-cC₃H₅ |
| A-268 | (E)-N-OCH₂CF₃ | 3-CF₃,5-cC₃H₅ |
| A-269 | (Z)-N-OCH₂C₆H₅ | 3-CF₃,5-cC₃H₅ |
| A-270 | (E)-N-OCH₂C₆H₅ | 3-CF₃,5-cC₃H₅ |
| A-271 | NOH | 3-Cl,5-[(1-CN)cC₃H₄] |
| A-272 | (Z)-N-OCH₃ | 3-Cl,5-[(1-CN)cC₃H₄] |
| A-273 | (E)-N-OCH₃ | 3-Cl,5-[(1-CN)cC₃H₄] |
| A-274 | (Z)-N-OC₂H₅ | 3-Cl,5-[(1-CN)cC₃H₄] |
| A-275 | (E)-N-OC₂H₅ | 3-Cl,5-[(1-CN)cC₃H₄] |
| A-276 | (Z)-N-OCH₂CF₃ | 3-Cl,5-[(1-CN)cC₃H₄] |
| A-277 | (E)-N-OCH₂CF₃ | 3-Cl,5-[(1-CN)cC₃H₄] |
| A-278 | (Z)-N-OCH₂C₆H₅ | 3-Cl,5-[(1-CN)cC₃H₄] |
| A-279 | (E)-N-OCH₂C₆H₅ | 3-Cl,5-[(1-CN)cC₃H₄] |
| A-280 | NOH | 3-CF₃,5-[(1-CN)cC₃H₄] |
| A-281 | (Z)-N-OCH₃ | 3-CF₃,5-[(1-CN)cC₃H₄] |
| A-282 | (E)-N-OCH₃ | 3-CF₃,5-[(1-CN)cC₃H₄] |
| A-283 | (Z)-N-OC₂H₅ | 3-CF₃,5-[(1-CN)cC₃H₄] |
| A-284 | (E)-N-OC₂H₅ | 3-CF₃,5-[(1-CN)cC₃H₄] |
| A-285 | (Z)-N-OCH₂CF₃ | 3-CF₃,5-[(1-CN)cC₃H₄] |
| A-286 | (E)-N-OCH₂CF₃ | 3-CF₃,5-[(1-CN)cC₃H₄] |
| A-287 | (Z)-N-OCH₂C₆H₅ | 3-CF₃,5-[(1-CN)cC₃H₄] |
| A-288 | (E)-N-OCH₂C₆H₅ | 3-CF₃,5-[(1-CN)cC₃H₄] |
| A-289 | NOH | 3-CF₃,5-[(2,2-Cl₂)-cC₃H₃] |
| A-290 | (Z)-N-OCH₃ | 3-CF₃,5-[(2,2-Cl₂)-cC₃H₃] |
| A-291 | (E)-N-OCH₃ | 3-CF₃,5-[(2,2-Cl₂)-cC₃H₃] |
| A-292 | (Z)-N-OC₂H₅ | 3-CF₃,5-[(2,2-Cl₂)-cC₃H₃] |
| A-293 | (E)-N-OC₂H₅ | 3-CF₃,5-[(2,2-Cl₂)-cC₃H₃] |
| A-294 | (Z)-N-OCH₂CF₃ | 3-CF₃,5-[(2,2-Cl₂)-cC₃H₃] |
| A-295 | (E)-N-OCH₂CF₃ | 3-CF₃,5-[(2,2-Cl₂)-cC₃H₃] |
| A-296 | (Z)-N-OCH₂C₆H₅ | 3-CF₃,5-[(2,2-Cl₂)-cC₃H₃] |
| A-297 | (E)-N-OCH₂C₆H₅ | 3-CF₃,5-[(2,2-Cl₂)-cC₃H₃] |
| A-298 | NOH | 3-OCF₃,5-cC₃H₅ |
| A-299 | (Z)-N-OCH₃ | 3-OCF₃,5-cC₃H₅ |
| A-300 | (E)-N-OCH₃ | 3-OCF₃,5-cC₃H₅ |
| A-301 | (Z)-N-OC₂H₅ | 3-OCF₃,5-cC₃H₅ |
| A-302 | (E)-N-OC₂H₅ | 3-OCF₃,5-cC₃H₅ |
| A-303 | (Z)-N-OCH₂CF₃ | 3-OCF₃,5-cC₃H₅ |
| A-304 | (E)-N-OCH₂CF₃ | 3-OCF₃,5-cC₃H₅ |
| A-305 | (Z)-N-OCH₂C₆H₅ | 3-OCF₃,5-cC₃H₅ |
| A-306 | (E)-N-OCH₂C₆H₅ | 3-OCF₃,5-cC₃H₅ |
| A-307 | NOH | 3,5-SO₂CH₃ |
| A-308 | (Z)-N-OCH₃ | 3,5-SO₂CH₃ |
| A-309 | (E)-N-OCH₃ | 3,5-SO₂CH₃ |
| A-310 | (Z)-N-OC₂H₅ | 3,5-SO₂CH₃ |
| A-311 | (E)-N-OC₂H₅ | 3,5-SO₂CH₃ |
| A-312 | (Z)-N-OCH₂CF₃ | 3,5-SO₂CH₃ |
| A-313 | (E)-N-OCH₂CF₃ | 3,5-SO₂CH₃ |
| A-314 | (Z)-N-OCH₂C₆H₅ | 3,5-SO₂CH₃ |
| A-315 | (E)-N-OCH₂C₆H₅ | 3,5-SO₂CH₃ |
| A-316 | NOH | 3,5-SO₂CF₃ |
| A-317 | (Z)-N-OCH₃ | 3,5-SO₂CF₃ |
| A-318 | (E)-N-OCH₃ | 3,5-SO₂CF₃ |
| A-319 | (Z)-N-OC₂H₅ | 3,5-SO₂CF₃ |
| A-320 | (E)-N-OC₂H₅ | 3,5-SO₂CF₃ |
| A-321 | (Z)-N-OCH₂CF₃ | 3,5-SO₂CF₃ |
| A-322 | (E)-N-OCH₂CF₃ | 3,5-SO₂CF₃ |
| A-323 | (Z)-N-OCH₂C₆H₅ | 3,5-SO₂CF₃ |
| A-324 | (E)-N-OCH₂C₆H₅ | 3,5-SO₂CF₃ |
| A-325 | NOH | 3-Cl,5-OC₆H₅ |
| A-326 | (Z)-N-OCH₃ | 3-Cl,5-OC₆H₅ |
| A-327 | (E)-N-OCH₃ | 3-Cl,5-OC₆H₅ |
| A-328 | (Z)-N-OC₂H₅ | 3-Cl,5-OC₆H₅ |
| A-329 | (E)-N-OC₂H₅ | 3-Cl,5-OC₆H₅ |
| A-330 | (Z)-N-OCH₂CF₃ | 3-Cl,5-OC₆H₅ |
| A-331 | (E)-N-OCH₂CF₃ | 3-Cl,5-OC₆H₅ |
| A-332 | (Z)-N-OCH₂C₆H₅ | 3-Cl,5-OC₆H₅ |
| A-333 | (E)-N-OCH₂C₆H₅ | 3-Cl,5-OC₆H₅ |
| A-334 | NOH | 3-CF₃,5-OC₆H₅ |
| A-335 | (Z)-N-OCH₃ | 3-CF₃,5-OC₆H₅ |
| A-336 | (E)-N-OCH₃ | 3-CF₃,5-OC₆H₅ |
| A-337 | (Z)-N-OC₂H₅ | 3-CF₃,5-OC₆H₅ |
| A-338 | (E)-N-OC₂H₅ | 3-CF₃,5-OC₆H₅ |
| A-339 | (Z)-N-OCH₂CF₃ | 3-CF₃,5-OC₆H₅ |
| A-340 | (E)-N-OCH₂CF₃ | 3-CF₃,5-OC₆H₅ |
| A-341 | (Z)-N-OCH₂C₆H₅ | 3-CF₃,5-OC₆H₅ |
| A-342 | (E)-N-OCH₂C₆H₅ | 3-CF₃,5-OC₆H₅ |
| A-343 | NOH | 3-CF₃,5-[O-(4-Cl-C₆H₄)] |
| A-344 | (Z)-N-OCH₃ | 3-CF₃,5-[O-(4-Cl-C₆H₄)] |
| A-345 | (E)-N-OCH₃ | 3-CF₃,5-[O-(4-Cl-C₆H₄)] |
| A-346 | (Z)-N-OC₂H₅ | 3-CF₃,5-[O-(4-Cl-C₆H₄)] |
| A-347 | (E)-N-OC₂H₅ | 3-CF₃,5-[O-(4-Cl-C₆H₄)] |
| A-348 | (Z)-N-OCH₂CF₃ | 3-CF₃,5-[O-(4-Cl-C₆H₄)] |
| A-349 | (E)-N-OCH₂CF₃ | 3-CF₃,5-[O-(4-Cl-C₆H₄)] |
| A-350 | (Z)-N-OCH₂C₆H₅ | 3-CF₃,5-[O-(4-Cl-C₆H₄)] |
| A-351 | (E)-N-OCH₂C₆H₅ | 3-CF₃,5-[O-(4-Cl-C₆H₄)] |
| A-352 | NOH | 3-SO₂(4-F-C₆H₄),5-Cl |
| A-353 | (Z)-N-OCH₃ | 3-SO₂(4-F-C₆H₄),5-Cl |
| A-354 | (E)-N-OCH₃ | 3-SO₂(4-F-C₆H₄),5-Cl |
| A-355 | (Z)-N-OC₂H₅ | 3-SO₂(4-F-C₆H₄),5-Cl |
| A-356 | (E)-N-OC₂H₅ | 3-SO₂(4-F-C₆H₄),5-Cl |
| A-357 | (Z)-N-OCH₂CF₃ | 3-SO₂(4-F-C₆H₄),5-Cl |
| A-358 | (E)-N-OCH₂CF₃ | 3-SO₂(4-F-C₆H₄),5-Cl |
| A-359 | (Z)-N-OCH₂C₆H₅ | 3-SO₂(4-F-C₆H₄),5-Cl |
| A-360 | (E)-N-OCH₂C₆H₅ | 3-SO₂(4-F-C₆H₄),5-Cl |
| A-361 | NOH | 3-SO₂(4-F-C₆H₄), |
| | | 5-[2,2-Cl₂-cC₃H₃] |
| A-362 | (Z)-N-OCH₃ | 3-SO₂(4-F-C₆H₄), |
| | | 5-[2,2-Cl₂-cC₃H₃] |
| A-363 | (E)-N-OCH₃ | 3-SO₂(4-F-C₆H₄), |
| | | 5-[2,2-Cl₂-cC₃H₃] |
| A-364 | (Z)-N-OC₂H₅ | 3-SO₂(4-F-C₆H₄), |
| | | 5-[2,2-Cl₂-cC₃H₃] |
| A-365 | (E)-N-OC₂H₅ | 3-SO₂(4-F-C₆H₄), |
| | | 5-[2,2-Cl₂-cC₃H₃] |
| A-366 | (Z)-N-OCH₂CF₃ | 3-SO₂(4-F-C₆H₄), |
| | | 5-[2,2-Cl₂-cC₃H₃] |
| A-367 | (E)-N-OCH₂CF₃ | 3-SO₂(4-F-C₆H₄), |
| | | 5-[2,2-Cl₂-cC₃H₃] |
| A-368 | (Z)-N-OCH₂C₆H₅ | 3-SO₂(4-F-C₆H₄), |
| | | 5-[2,2-Cl₂-cC₃H₃] |
| A-369 | (E)-N-OCH₂C₆H₅ | 3-SO₂(4-F-C₆H₄), |
| | | 5-[2,2-Cl₂-cC₃H₃] |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emulsifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are quaternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grapefruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes, sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aerosol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plu-tella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Mosquitoes (Diptera), e.g. Aedes aegypti, A. albopictus, A. vexans, Anastrepha ludens, Anopheles maculipennis, A. crucians, A. albimanus, A. gambiae, A. freeborni, A. leucosphyrus, A. minimus, A. quadrimaculatus;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne sp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets, or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition, the compounds I may be transdermally administered to animals. For parenteral administration, the dosage form chosen should provide the animal with 0.01 mg/kg to 100 mg/kg of animal body weight per day of the compounds 1.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds 1. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending, or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

The compounds were characterized by melting point determination, by NMR spectroscopy or by the mass-to-charge ratio ([m/z]) and retention time (RT; [min]), as determined by mass spectrometry (MS) coupled with HPLC analysis (HPLC-MS=high performance liquid chromatography-coupled mass spectrometry) or LC analysis (LC-MS=liquid chromatography-coupled mass spectrometry).

Method A: HPLC: Shimadzu Nexera UHPLC+Shimadzu LCMS-2020, ESI; Column: Phenomenex Kinetex 1.7 μm XB-C18 100A, 2.1×50 mm; Mobile phase: A: water+0.1% TFA; B: ACN;

Temperature: 60° C.; Gradient: 5% B to 100% B in 1.5 min; 100% B 0.25 min; Flow: 0.8 mL/min to 1.0 mL/min in 1.51 min; MS: ESI positive; Mass range (m/z): 100-700.

Method B: LC: Shimadzu LC-30AD, ESI; Column: Kinetex EVO C₁₈.5 μm 2.1×30 mm; Mobile phase: A: water+0.04% TFA; B: ACN+0.02% TFA; Temperature: 40° C.; Gradient: 5% B to 100% B in 2.5 min; 100% B to 5% B in 0.02 min; 5% B for 0.5 min; Flow: 0.8 mL/min; MS: ESI positive; Mass range: 100-2000.

### Example 1: preparation of N-[1-[3-[N′-hydroxycarbamimidoyl]pyrazin-2-yl]ethyl]-3,5-bis(trifluoromethyl)benzamide (I-3)

### Step 1: Preparation of 1-(3-chloropyrazin-2-yl)ethanamine

To a mixture of 1-(3-chloropyrazin-2-yl)ethanone (10 g, 63.8 mmol) and NH₄OAc (60.4 g, 957.8 mmol) in EtOH (500 mL) were added NaBH₃CN (12.04 g, 191.4 mmol) and NH₃/MeOH (150 mL, 7N) at 30° C. The mixture was stirred at 50° C. until completion was determined by LCMS (16 h). The reaction mixture was quenched with H₂O (100 mL) and concentrated to remove MeOH and EtOH. 6N aq. NaOH was added to adjust the pH of the residue to 11 and the resulting mixture was extracted with EtOAc (3×100 mL). The combined organic layers were dried over Na₂SO₄, filtered, and concentrated. The crude product was purified by preparative HPLC (NH₄HCO₃) to deliver 1-(3-chloropyrazin-2-yl)ethanamine (10 g, 50% yield) as a white solid.

¹H-NMR (400 MHz, DMSO-d₆): δ=8.70 (d, J=2.4 Hz, 1H), 8.47 (d, J=2.5 Hz, 1H), 4.50 (q, J=6.7 Hz, 1H), 1.37 (d, J=6.8 Hz, 3H).

### Step 2: preparation of N-[1-(3-chloropyrazin-2-yl)ethyl]-3,5-bis(trifluoromethyl)benzamide

To a mixture of 3,5-bis(trifluoromethyl)benzoic acid (19.65 g, 76.14 mmol) and chloro-N,N,N′,N′-tetramethylformamidinium hexafluorophosphate (21.36 g, 76.14 mmol) in MeCN (200 mL) were added N-methylimidazole (16.7 g, 203.2 mmol) and 1-(3-chloropyrazin-2-yl)ethanamine (8.0 g, 50.8 mmol) at 30° C. The reaction mixture was stirred until completion was determined by TLC (PE:EtOAc=3:1, Rf=0.45; 16 h). The reaction mixture was quenched with H₂O (50 mL) and extracted with EtOAc (3×100 mL). The combined organic layers were dried over Na₂SO₄, filtered, and concentrated. The crude product was purified by chromatographic column on silica gel (eluent: 12% EtOAc in PE) to deliver N-[1-(3-chloropyrazin-2-yl)ethyl]-3,5-bis(trifluoromethyl)benzamide (13 g, 43% yield) as a yellow solid.

¹H NMR (400 MHz, DMSO-d₆): δ=9.53 (d, J=6.8 Hz, 1H), 8.65 (d, J=2.5 Hz, 1H), 8.55 (s, 2H), 8.46 (d, J=2.5 Hz, 1H), 8.33 (s, 1H), 5.53 (dq, J=6.8 Hz, J=6.9 Hz, 1H), 1.56 (d, J=6.9 Hz, 3H).

### Step 3: preparation of N-[1-(3-cyanopyrazin-2-yl)ethyl]-3,5-bis(trifluoromethyl)benzamide

To a solution of N-[1-(3-chloropyrazin-2-yl)ethyl]-3,5-bis(trifluoromethyl)benzamide (1.0 g, 2.51 mmol) in DMF (10 mL) were added Zn(CN)₂ (330 mg, 2.76 mmol) and Pd(PPh₃)₄ (350 mg, 0.30 mmol) at 20° C., and the resulting mixture was purged with N₂ for 3 min. The vial was sealed and subjected to microwave irradiation (Biotage Smith Synthesis, 130° C., 10 min). Completion was determined by TLC (PE:EtOAc=3:1, Rf=0.4). The reaction mixture was filtered through a celite pad, the filter cake was washed with EtOAc (50 mL), and the filtrate was concentrated. The crude product was purified by chromatographic column on silica gel (eluent: 17% EtOAc in PE) to deliver N-[1-(3-cyanopyrazin-2-yl)ethyl]-3,5-bis(trifluoromethyl)benzamide (2.6 g, 45% yield) as a yellow solid.

¹H-NMR (400 MHz, CDCl₃): δ 8.77 (d, J=2.3 Hz, 1H), 8.70 (d, J=2.4 Hz, 1H), 8.28 (s, 2H), 8.04 (s, 1H), 7.37-7.28 (m, 1H), 5.84-5.73 (m, 1H), 1.74 (d, J=6.8 Hz, 3H).

### Step 4: preparation of N-[1-[3-[N′-hydroxycarbamimidoyl]pyrazin-2-yl]ethyl]-3,5-bis(trifluoromethyl)benzamide (I-3)

To a solution of N-[1-(3-cyanopyrazin-2-yl)ethyl]-3,5-bis(trifluoromethyl)benzamide (1.1 g, 2.8 mmol) in EtOH (20 mL) were added HONH₂·HCl (238 mg, 3.36 mmol) and triethylamine (575 mg, 5.6 mmol) at 30° C. The resulting mixture was stirred at 80° C. for 2.5 h, until completion was determined by TLC (PE:EtOAc=3:1, Rf=0.3). The reaction mixture was quenched with H₂O (10 mL) and extracted with EtOAc (3×20 mL). The combined organic layers were dried over Na₂SO₄, filtered, and concentrated. The crude product was purified by chromatographic column on silica gel (25% EtOAc in PE) to deliver N-[1-[3-[N′-hydroxycarbamimidoyl]pyrazin-2-yl]ethyl]-3,5-bis(trifluoromethyl)benzamide (I-3, 600 mg, 51% yield) as a white solid.

¹H-NMR (400 MHz, DMSO-d₆) 5=10.21 (s, 1H), 9.42 (d, J=7.2 Hz, 1H), 8.62 (d, J=2.4 Hz, 1H), 8.57 (d, J=2.3 Hz, 1H), 8.54 (s, 2H), 8.30 (s, 1H), 6.12 (dq, J=6.8 Hz, J=7.2 Hz, 1H), 5.96 (s, 2H), 1.54 (d, J=6.8 Hz, 3H).

LCMS: 421.1 (desired); 422.0 (observed).

### Example 2: preparation of N-[1-[3-[N′-methoxycarbamimidoyl]pyrazin-2-yl]ethyl]-3,5-bis(trifluoromethyl)benzamide (I-5)

To a solution of N-[1-[3-[N′-hydroxycarbamimidoyl]pyrazin-2-yl]ethyl]-3,5-bis(trifluoromethyl)benzamide (I-3, 600 mg, 1.42 mmol) in MeOH (4.8 mL) was added a solution of KOH (72 mg, 1.28 mmol) in H₂O (1.2 mL). Next, Mel (182 mg, 1.28 mmol) was added dropwise at 30° C. The reaction was stirred for 16 h, until completion was determined by TLC (PE:EtOAc=3:1, Rf=0.4). The reaction mixture was quenched with H₂O (10 mL) and the pH of the resulting mixture was adjusted to 7 by addition of 1N aq. HCl. The resulting mixture was extracted with EtOAc (3×30 mL). The combined organic layers were dried over Na₂SO₄, filtered, and concentrated. The crude product was purified by chromatographic column on silica gel (20% EtOAc in PE) to deliver N-[1-[3-[N′-methoxycarbamimidoyl]pyrazin-2-yl]ethyl]-3,5-bis(trifluoromethyl)benzamide (I-5, 145 mg, 23% yield) as a yellow solid.

¹H-NMR (400 MHz, DMSO-d₆) 5=9.43 (d, J=7.0 Hz, 1H), 8.65 (d, J=2.3 Hz, 1H), 8.58 (d, J=2.3 Hz, 1H), 8.53 (s, 2H), 8.30 (s, 1H), 6.21 (s, 2H), 5.98 (dq, J=6.8 Hz, J=7.0 Hz, 1H), 3.80 (s, 3H), 1.58 (d, J=6.8 Hz, 3H).

LCMS: 435.1 (desired); 436.1 (observed).

### Example 3: preparation of N-[1-[3-[N-methoxy-C-methyl-carbonimidoyl]pyrazin-2-yl]ethyl]-3,5-bis(trifluoromethyl)benzamide (I-1)

### Step 1: preparation of N-[1-[3-(1-ethoxyvinyl)pyrazin-2-yl]ethyl]-3,5-bis(trifluoromethyl)benzamide

To a stirred solution of N-[1-(3-chloropyrazin-2-yl)ethyl]-3,5-bis(trifluoromethyl)benzamide (1 g, 2.5 mmol) in toluene (100 mL) were added tributyl(1-ethoxyvinyl)stannane (1.1 g, 3.0 mmol) and Pd(PPh₃)₂Cl₂ (0.1 g, 0.25 mmol) at 25° C. The reaction mixture was stirred for 12 h at 110° C. under an atmosphere of N₂, at which time completion was determined by TLC (PE:EtOAc=3:1). The reaction mixture was cooled down to 25° C. and aq. sat. KF (100 mL) was added. The mixture was stirred for 30 min, then filtered through a celite pad. The filtrate was extracted with EtOAc (3×40 mL). The combined organic layers were washed with brine (60 mL), dried over Na₂SO₄, and concentrated to furnish N-[1-[3-(1-ethoxyvinyl)pyrazin-2-yl]ethyl]-3,5-bis(trifluoromethyl)benzamide (1.0 g, crude) as a thick yellow oil. The crude product was employed in the next step without further purification.

¹H-NMR (400 MHz, CDCl₃) δ=8.54 (s, 2H), 8.27 (s, 2H), 8.01 (s, 1H), 7.71 (br d, J=7.5 Hz, 1H), 5.86-6.02 (m, 1H), 4.87 (d, J=2.6 Hz, 1H), 4.60 (d, J=2.6 Hz, 1H), 3.96-4.17 (m, 2H), 1.59 (d, J=6.5 Hz, 3H), 1.50 (t, J=7.0 Hz, 3H).

### Step 2: preparation of N-[1-(3-acetylpyrazin-2-yl)ethyl]-3,5-bis(trifluoromethyl)benzamide

To a stirred solution of N-[1-[3-(1-ethoxyvinyl)pyrazin-2-yl]ethyl]-3,5-bis(trifluoromethyl)benzamide (4.3 g, 9.93 mmol) in THF (60 mL) was added aq. HCl (2M, 60 mL) dropwise at 0° C. The mixture was stirred for 2 h at 20° C., until completion was determined by TLC (PE:EtOAc=3:1). The reaction mixture was diluted with H₂O (100 mL), extracted with EtOAc (3×50 mL). The combined organic layers were washed with brine (100 mL), dried over Na₂SO₄, and concentrated to deliver N-[1-(3-acetylpyrazin-2-yl)ethyl]-3,5-bis(trifluoromethyl)benzamide (1.5 g, 37% yield) as a white solid.

¹H-NMR (400 MHz, CDCl₃) 5=8.71 (d, J=2.3 Hz, 1H), 8.63 (d, J=2.3 Hz, 1H), 8.25 (s, 2H), 8.01 (s, 1H), 7.60 (br d, J=7.8 Hz, 1H), 6.03-6.17 (m, 1H), 2.79 (s, 3H), 1.65 (d, J=6.8 Hz, 3H).

### Step 3: preparation of N-[1-[3-[N-methoxy-C-methyl-carbonimidoyl]pyrazin-2-yl]ethyl]-3,5-bis(trifluoromethyl)benzamide (I-1)

To a stirred solution of N-[1-(3-acetylpyrazin-2-yl)ethyl]-3,5-bis(trifluoromethyl)benzamide (400 mg, 0.987 mmol) in EtOH (10 mL) were added NaOAc (0.162 g, 1.97 mmol) and MeONH₂—HCl (0.082 g, 0.987 mmol) at 20° C. The reaction mixture was stirred for 12 h at 50° C., at which time completion was observed by LCMS. The reaction mixture was diluted with H₂O (10 mL) and extracted with EtOAc (3×30 mL). The combined organic layers were washed with brine (10 mL), dried over Na₂SO₄, and concentrated. The residue was purified by chromatographic column on silica gel (PE:EtOAc=100:0 to 82:18) to deliver N-[1-[3-[N-methoxy-C-methyl-carbonimidoyl]pyrazin-2-yl]ethyl]-3,5-bis(trifluoromethyl)benzamide (I-1, 0.258 g, 60% yield) as a white solid.

¹H-NMR (400 MHz, CDCl₃) δ=8.57 (d, J=2.4 Hz, 1H), 8.53 (d, J=2.4 Hz, 1H), 8.01 (s, 1H), 8.26 (s, 2H), 7.66 (br d, J=7.8 Hz, 1H), 6.07 (dq, J=7.8, 6.7 Hz, 1H), 2.36 (s, 3H), 4.12 (s, 3H), 1.65 (d, J=6.50 Hz, 3H).

LCMS: 434.12 (calculated); 435.0 (observed).

### Example 4: preparation of 3-(1-cyanocyclopropyl)-N-(cyclopropylmethyl)-N-[1-[3-(N-methoxy-C-methyl-carbonimidoyl)pyrazin-2-yl]ethyl]-5-(trifluoromethyl)benzamide (I-9)

### Step 1: preparation of 3-(1-cyanocyclopropyl)-N-(cyclopropylmethyl)-N-[1-[3-(N-methoxy-C-methyl-carbonimidoyl)pyrazin-2-yl]ethyl]-5-(trifluoromethyl)benzamide

To a stirred solution of 3-(1-cyanocyclopropyl)-N-[1-[3-(N-methoxy-C-methyl-carbonimidoyl)pyrazin-2-yl]ethyl]-5-(trifluoromethyl)benzamide (150 mg, 0.348 mmol, prepared via the same synthetic route as 1-1) in DMF (5 mL) was added NaH (28 mg, 0.696 mmol) in portions at 0° C. The reaction mixture was stirred for 30 min at 0° C. and then heated to 80° C. A solution of bromomethylcyclopropane (70 mg, 0.522 mmol) in DMF (5 mL) was added dropwise at 80° C. and stirred at this temperature for 12 h. The reaction mixture was cooled to RT, quenched with NH₄Cl (aq. 10 mL) and extracted with EtOAc (3×10 mL). The combined organic layers were washed with brine (4×5 mL), dried over Na₂SO₄, filtered and concentrated under reduced pressure. The residue was purified by preparative-HPLC (NH₄HCO₃) to deliver 3-(1-cyanocyclopropyl)-N-(cyclopropylmethyl)-N-[1-[3-(N-methoxy-C-methyl-carbonimidoyl)pyrazin-2-yl]ethyl]-5-(trifluoromethyl)benzamide (I-9, 50 mg, 15% yield) as a yellow syrup.

1H-NMR (400 MHz, CDCl3) δ=8.64 (d, J=2.45 Hz, 1H), 8.61 (d, J=2.45 Hz, 1H), 7.64 (s, 1H), 7.46 (s, 1H), 7.42 (s, 1H), 5.96 (br s, 1H), 3.80 (s, 3H), 3.35-3.47 (m, 1H), 3.18-3.28 (m, 1H), 2.10 (s, 3H), 1.81-1.85 (m, 2H), 1.69 (d, J=6.97 Hz, 3H), 1.59-1.63 (m, 2H), 0.85-0.90 (m, 1H), 0.31-0.43 (m, 2H), 0.13-0.10 (m, 2H).

### Example 5: preparation of 3-chloro-N-(cyclopropylmethyl)-5-(4-fluorophenyl)sulfonyl-N-[1-[3-(N-methoxy-C-methyl-carbonimidoyl)pyrazin-2-yl]ethyl]benzamide (I-17)

### Step 1: preparation of 1-(3-chloropyrazin-2-yl)-N-(cyclopropylmethyl)ethanamine

To a stirred solution of 1-(3-chloropyrazin-2-yl)ethenone (200 mg, 3.2 mmol) in THF (20 mL) at RT were added cyclopropylmethanamine (109 mg, 0.364 mmol) and Ti(i-PrO)₄ (364 mg; 1.282 mmol). The reaction mixture was stirred for 12 h at RT and then stirred at 50° C. for 4 h. TLC (PE:EtOAc=1:1) showed complete conversion. The reaction mixture was cooled to RT, then EtOH (10 mL) was added and NaBH₃CN (121 mg, 1.923 mmol) was added in portions. TLC (PE:EtOAc=1:1) showed the reaction was completed after 12 h at RT. The reaction mixture was quenched with water (30 mL), filtered and the filtrate was extracted with EtOAc (3×20 mL). The organic layers were washed with brine (20 mL), dried over Na₂SO₄ and concentrated under reduced pressure. The residue was purified by chromatographic column on silica gel (PE:EtOAc=100:0 to 16:84) to deliver 1-(3-chloropyrazin-2-yl)-N-(cyclopropylmethyl)ethanamine (94 mg, 35% yield) as a yellow syrup.

1H-NMR (400 MHz, CDCl3) δ=8.50 (d, J=2.38 Hz, 1H), 8.25 (d, J=2.38 HZ, 1H), 4.37 (q, J=6.65 Hz, 1H), 2.51 (dd, J=11.73, 6.21 Hz, 1H), 2.05 (dd, J=11.67, 7.53 Hz, 1H), 1.38 (d, J=6.65 Hz, 3H), 0.83-1.00 (m, 1H), 0.43 (td, J=7.87, 2.45 Hz, 2H), 0.06-0.14 (m, 2H).

### Step 2: preparation of 3-chloro-N-[1-(3-chloropyrazin-2-yl)ethyl]-N-(cyclopropylmethyl)-5-(4-fluorophenyl)sulfonyl-benzamide

To a stirred solution of 3-chloro-5-(4-fluorophenyl)sulfonyl-benzoic acid (100 mg, 0.318 mmol) in ACN (5 mL) were added chloro-N,N,N′,N′-tetramethylformamidinium-hexafluorophosphate (134 mg, 0.477 mmol), N-methylimidazole (78 mg; 0.995 mmol) and 1-(3-chloropyrazin-2-yl)-N-(cyclopropylmethyl)ethanamine (81 mg, 0.382 mmol) at RT. LC-MS showed the reaction was completed after 12 h. The reaction mixture was quenched with water (10 mL) and extracted with EtOAc (3×8 mL). The combined organic layers were washed with brine (10 mL) dried over Na₂SO₄, filtered and concentrated under reduced pressure. The residue was purified by preparative TLC (PE:EtOAc=1:1) to deliver 3-chloro-N-[1-(3-chloropyrazin-2-yl)ethyl]-N-(cyclopropylmethyl)-5-(4-fluorophenyl)sulfonyl-benzamide (50 mg, 31% yield) as a yellow oil.

1H-NMR (400 MHz, CDCl3) δ=8.51 (d, J=2.38 Hz, 1H), 8.27 (d, J=2.38 Hz, 1H), 7.94-8.00 (m, 2H), 7.88 (br s, 1H), 7.61 (s, 1H), 7.23 (br t, J=8.50 Hz, 2H), 4.42 (q, J=6.71 Hz, 1H), 2.56 (dd, J=11.76, 6.25 Hz, 1H), 2.09-2.13 (m, 1H), 1.43 (d, J=6.63 Hz, 3H), 0.93-0.98 (m, 1H), 0.44-0.48 (m, 2H), 0.21-0.33 (m, 1H), 0.05-0.10 (m, 1H).

### Step 3: preparation of 3-chloro-N-(cyclopropylmethyl)-N-[1-[3-(1-ethoxyvinyl)pyrazin-2-yl]ethyl]-5-(4-fluorophenyl)sulfonyl-benzamide

To a stirred solution of 3-chloro-N-[1-(3-chloropyrazin-2-yl)ethyl]-N-(cyclopropylmethyl)-5-(4-fluorophenyl)sulfonyl-benzamide (300 mg, 0.662 mmol) in toluene (8 mL) were added tributyl(1-ethoxyvinyl)stannane (287 mg, 0.795 mmol) and Pd(PPh₃)₂Cl₂ (100 mg, cat.) at RT. The sealed vial was irradiated in the microwave for 1 h at 130° C. TLC (PE:EtOAc=3:1) showed the reaction was completed. The reaction mixture was concentrated under reduced pressure. The residue was diluted with aq. KF (10 mL) and stirred for 30 min. The reaction mixture was filtered through a celite pad and the filtrate was extracted with EtOAc (3×30 mL). The combined organic layers were washed with brine (6 ml), dried over Na₂SO₄, filtered and concentrated under reduced pressure to deliver 3-chloro-N-(cyclopropylmethyl)-N-[1-[3-(1-ethoxyvinyl)pyrazin-2-yl]ethyl]-5-(4-fluorophenyl)sulfonyl-benzamide (4.3 g, crude) as a yellow oil. The crude product was employed in the next step without further purification.

### Step 4: preparation of N-[1-(3-acetylpyrazin-2-yl)ethyl]-3-chloro-N-(cyclopropylmethyl)-5-(4-fluorophenyl)sulfonyl-benzamide

To a stirred solution of 3-chloro-N-(cyclopropylmethyl)-N-[1-[3-(1-ethoxyvinyl)pyrazin-2-yl]ethyl]-5-(4-fluorophenyl)sulfonyl-benzamide (1.3 g, 2.39 mmol) in THF (20 mL) was added HCl (aq., 2N, 10 mL) at RT. The reaction mixture was stirred for 4 h at RT. TLC (PE:EtOAc=1:1) showed the reaction was completed. The reaction mixture was diluted with water (10 mL) and extracted with EtOAc (3×20 mL). The combined organic layers were washed with brine (10 mL), dried over Na₂SO₄, filtered and concentrated under reduced pressure. The residue was purified by chromatographic column on silica gel (PE:EtOAc=100:0 to 35:65) to deliver N-[1-(3-acetylpyrazin-2-yl)ethyl]-3-chloro-N-(cyclopropylmethyl)-5-(4-fluorophenyl)sulfonyl-benzamide (500 mg, 70% yield) as a yellow solid.

### Step 5: preparation of 3-chloro-N-(cyclopropylmethyl)-5-(4-fluorophenyl)sulfonyl-N-[1-[3-(N-methoxy-C-methyl-carbonimidoyl)pyrazin-2-yl]ethyl]benzamide

To a stirred solution of N-[1-(3-acetylpyrazin-2-yl)ethyl]-3-chloro-N-(cyclopropylmethyl)-5-(4-fluorophenyl)sulfonyl-benzamide (500 mg, 0.971 mmol) in EtOH (5 mL) were added NaOAc (159 mg, 1.94 mmol) and O-methylhydroxylamine hydrochloride (122 mg, 1.45 mmol) at RT. The reaction mixture was heated to 50° C. and stirred for 12 h. LC-MS showed the reaction was completed. The reaction mixture was concentrated under reduced pressure, quenched with water (10 ml) and extracted with EtOAc (3×10 mL). The combined organic layers were washed with brine (5 mL), dried over Na₂SO₄, filtered and concentrated under reduced, pressure. The residue was purified by chromatographic column on silica gel (PE:EtOAc=100:0 to 50:50) to deliver 3-chloro-N-(cyclopropylmethyl)-5-(4-fluorophenyl)sulfonyl-N-[1-[3-(N-methoxy-C-methyl-carbonimidoyl)pyrazin-2-yl]ethyl]benzamide (I-17, 268 mg, 51% yield) as a yellow solid.

1H-NMR (400 MHz, CDCl3) δ=8.64 (s, 1H), 8.61 (s, 1H), 8.03-8.09 (m, 3H), 7.64 (br s, 1H), 7.58 (s, 1H), 7.45 (br t, J=8.58 Hz, 2H), 5.94 (br d, J=1.91 Hz, 1H), 3.73 (br s, 3H), 3.13-3.42 (m, 2H), 2.03-2.14 (m, 3H), 1.65 (br d, J=6.79 Hz, 3H), 0.65-0.97 (m, 1H), 0.23-0.41 (m, 2H), −0.28-0.09 (m, 2H).

### Example 6 preparation of N-[1-[3-(N-ethyl-N′-hydroxy-N-methyl-carbamimidoyl)pyrazin-2-yl]ethyl]-3,5-bis(trifluoromethyl)benzamide (I-170)

### Step 1: preparation of 3-[1-[[3,5-bis(trifluoromethyl)benzoyl]amino]ethyl]-N-hydroxy-pyrazine-2-carboximidoyl chloride

To a stirred solution of N-[1-[3-(N′-hydroxycarbamimidoyl)pyrazin-2-yl]ethyl]-3,5-bis(trifluoromethyl)benzamide (3 g, 7.1 mmol) in HCl (aq., 6N, 30 mL) at 0° C. was added dropwise a solution of NaNO₂ (735 mg, 10.6 mmol) in water (2 mL). The reaction mixture was stirred for 3 h at RT. LCMS showed the reaction was completed. The reaction mixture was extracted with EtOAc (3×50 mL). The combined organic layers were dried over Na₂SO₄, filtered and concentrated under reduced pressure to deliver 3-[1-[[3,5-bis(trifluoromethyl)benzoyl]amino]ethyl]-N-hydroxypyrazine-2-carboximidoyl chloride (3 g, crude) as a yellow solid. The crude product was employed in the next step without further purification

### Step 2: preparation of N-[1-[3-(N-ethyl-N′-hydroxy-N-methyl-carbamimidoyl)pyrazin-2-yl]ethyl]-3,5-bis(trifluoromethyl)benzamide

To a stirred solution of 3-[1-[[3,5-bis(trifluoromethyl)benzoyl]amino]ethyl]-N-hydroxy-pyrazine-2-carboximidoyl chloride (500 mg, 1.1 mmol) in THF (10 mL) was added dropwise N-methylethanamine (80 mg, 1.4 mmol) at RT. The reaction mixture was stirred for 16 h at RT under N₂ atmosphere. TLC (PE:EtOAc=1:1) showed the reaction was completed. The reaction mixture was concentrated under reduced pressure and the crude was purified by chromatographic column on silica gel (PE:EtOAc=1:1) to deliver N-[1-[3-(N-ethyl-N′-hydroxy-N-methyl-carbamimidoyl)pyrazin-2-yl]ethyl]-3,5-bis(trifluoromethyl)benzamide (I-170, 300 mg, 59% yield) as a yellow syrup.

1H-NMR (400 MHz, CDCl3) δ=8.86 (d, J=1.63 Hz, 1H), 8.74 (s, 1H), 8.47 (s, 2H), 8.18 (s, 1H), 5.26-5.47 (m, 1H), 3.37 (br d, J=7.25 Hz, 2H), 1.72 (d, J=6.88 Hz, 3H), 1.27 (t, J=7.13 Hz, 3H).

With appropriate modification of the starting materials, the procedures given in the synthesis descriptions were used to obtain further compounds I. The compounds obtained in this manner are listed in the table that follows, together with physical data.

TABLE I 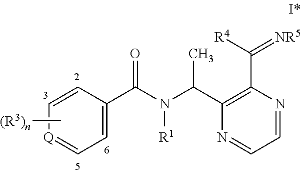
compounds of formula I* (R² = CH₃)

| | | | | | | | phys. | |
|---|---|---|---|---|---|---|---|---|
| | | | | | | | data | |
| | | | | | | | (HPLC | |
| | | | | | | | RT | M + H |
| No. | R¹ | Q | R³ | NR⁵ | R⁴ | Method | [min]) | [m/z] |
| I-1 | H | CH | 3,5-(CF₃)₂ | NOCH₃ | CH₃ | A | 1.309 | 435.3 |
| I-2 | CH₂cC₃H₅ | CH | 3,5-(CF₃)₂ | NOH | NH₂ | B | 1.544 | 476.1 |
| I-3 | H | CH | 3,5-(CF₃)₂ | NOH | NH₂ | B | 1.382 | 422.0 |
| I-4 | CH₂cC₃H₅ | CH | 3,5-(CF₃)₂ | NOCH₃ | NH₂ | B | 1.744 | 490.2 |
| I-5 | H | CH | 3,5-(CF₃)₂ | NOCH₃ | NH₂ | B | 1.600 | 436.1 |
| I-6 | CH₂cC₃H₅ | CH | 3,5-(CF₃)₂ | NOCH₃ | NHCH₃ | B | 1.699 | 504.1 |
| I-7 | H | CH | 3-(1-CN-cC₃H₅), | NOCH₃ | CH₃ | B | 1.624 | 432.1 |
| | | | 5-CF₃ | | | | | |
| I-8 | CH₂cC₃H₅ | CH | 3-Cl,5-(SO₂CH₃) | NOCH₃ | CH₃ | B | 1.581 | 465.1 |
| I-9 | CH₂cC₃H₅ | CH | 3-(1-CN-c-C₃H₅), | NOCH₃ | CH₃ | A | 1.339 | 486.3 |
| | | | 5-CF₃ | | | | | |
| I-10 | H | CH | 3,5-(CF₃)₂ | N—NH—C(═O)—NHCH₃ | CH₃ | A | 1.113 | 476.8 |
| I-11 | H | CH | 3,5-(CF₃)₂ | NOCH₂CH₃ | CH₃ | A | 1.365 | 448.9 |
| I-12 | H | CH | 3,5-(CF₃)₂ | NOH | CH₃ | A | 1.139 | 420.8 |
| I-13 | H | CH | 3,5-(CF₃)₂ | NOCH₃ | H | A | 1.248 | 421.1 |
| I-14 | H | CH | 3,5-(CF₃)₂ | NOH | H | A | 1.115 | 407.1 |
| I-15 | H | CH | 3,5-(CF₃)₂ | N—NH—C(═O)—NHCH₃ | H | A | 1.054 | 462.9 |
| I-16 | H | CH | 3-Cl,5-SO₂— | NOCH₃ | CH₃ | A | 1.221 | 491 |
| | | | (4-F—C₆H₄) | | | | | |
| I-17 | CH₂cC₃H₅ | CH | 3-Cl,5-SO₂— | NOCH₃ | CH₃ | A | 1.368 | 545.0 |
| | | | (4-F—C₆H₄) | | | | | |
| I-18 | CH₂cC₃H₅ | CH | 3-OCHF₂, | NOCH₃ | CH₃ | A | 1.383 | 487.1 |
| | | | 5-CF₃ | | | | | |
| I-19 | H | CH | 3-OCHF₂, | NOCH₃ | CH₃ | A | 1.265 | 432.8 |
| | | | 5-CF₃ | | | | | |
| I-20 | H | CH | 3,5-(CF₃)₂ | N—NHC(O)NH(2,4-Cl₂—C₆H₃) | H | A | 1.367 | 593.1 |
| I-21 | H | CH | 3,5-(CF₃)₂ | N—N(CH₃)C(O)NHC₂H₅ | H | A | 1.192 | 490.1 |
| I-22 | H | CH | 3,5-(CF₃)₂ | N—NHC(O)NHCH₂cC₃H₅ | H | A | 1.180 | 503.2 |
| I-23 | H | CH | 3,5-(CF₃)₂ | 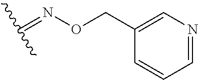 [SMILES: *=NOCc1cccnc1] | H | A | 0.973 | 498.2 |
| I-24 | H | CH | 3,5-(CF₃)₂ | N—OCH₂CH₂OCH(CH₃)₂ | H | A | 1.319 | 493.2 |
| I-25 | H | CH | 3,5-(CF₃)₂ | N—OC(CH₃)O(2-F—C₆H₄) | H | A | 1.401 | 567.2 |
| | | | | | | | | (M + |
| | | | | | | | | Na)⁺ |
| I-26 | H | CH | 3,5-(CF₃)₂ | N—OCH₂C(O)NH₂ | H | A | 1.271 | 464.2 |
| I-27 | H | CH | 3,5-(CF₃)₂ | N—NC(O)NH(2-CH₃—C₆H₄) | CH₃ | A | 1.289 | 553.2 |
| I-28 | H | CH | 3,5-(CF₃)₂ | 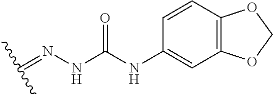 [SMILES: *=NNC(=O)Nc1ccc2c(c1)OCO2] | CH₃ | A | 1.259 | 583.2 |
| I-29 | H | CH | 3,5-(CF₃)₂ | N—NHC(O)NH(2,4-F₂—C₆H₃) | CH₃ | A | 1.371 | 575.2 |
| I-30 | H | CH | 3,5-(CF₃)₂ | N—NHC(O)NHCH₂cC₃H₅ | CH₃ | A | 1.230 | 517.2 |
| I-31 | H | CH | 3,5-(CF₃)₂ | N—OCH(CH₂OH)cC₅H₇ | CH₃ | A | 1.397 | 533.3 |
| I-32 | H | CH | 3,5-(CF₃)₂ | N—OCH₂(4-C(CH₃)₃-C₆H₄) | CH₃ | A | 1.599 | 567.3 |
| I-33 | H | CH | 3,5-(CF₃)₂ | N—OCH₂(3-Br,5-F—C₆H₃) | CH₃ | A | 1.526 | 609.1 |
| I-34 | CH₃ | CH | 3,5-(CF₃)₂ | NOH | NHCH₃ | B | 1.292 | 450.1 |
| I-35 | H | CH | 3,5-(CF₃)₂ | NOCH₃ | NHCH₃ | B | 1.507 | 450.1 |
| I-36 | H | N | 3-(SO₂CH₃), | NOCH₃ | CH₃ | B | 1.42 | 446.1 |
| | | | 5-CF₃ | | | | | |
| I-37 | H | CH | 3,5-(CF₃)₂ | N—NC(O)NH[4-N(CH₃)₂-C₆H₄] | H | A | 0.957 | 568.2 |
| I-38 | H | CH | 3,5-(CF₃)₂ | 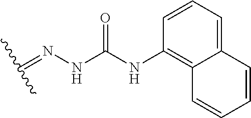 [SMILES: *=NNC(=O)Nc1cccc2ccccc12] | H | A | 1.272 | 575.2 |
| I-39 | H | CH | 3,5-(CF₃)₂ | 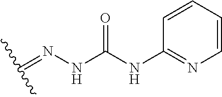 [SMILES: *=NNC(=O)Nc1ccccn1] | H | A | 0.975 | 526.2 |
| I-40 | H | CH | 3,5-(CF₃)₂ | N—NHC(O)NHCH₂C₆H₅ | H | A | 1.223 | 539.2 |
| I-41 | H | CH | 3,5-(CF₃)₂ | N—NHC(O)NHCH₂[3-C(CH₃)₃C₆H₄] | H | A | 1.293 | 607.2 |
| I-42 | H | CH | 3,5-(CF₃)₂ | N—NHC(O)NHCH₂(4-OCHF₂)C₆H₄ | H | A | 1.261 | 591.2 |
| I-43 | H | CH | 3,5-(CF₃)₂ | N—NHC(O)NHCH₂(4-F—C₆H₄) | H | A | 1.247 | 543.2 |
| I-44 | H | CH | 3,5-(CF₃)₂ | 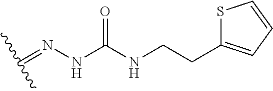 [SMILES: *=NNC(=O)NCCc1cccs1] | H | A | 1.225 | 559.1 |
| I-45 | H | CH | 3,5-(CF₃)₂ | 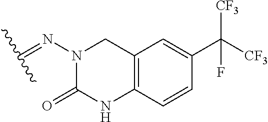 [SMILES: *=NN1Cc2cc(C(F)(C(F)(F)F)C(F)(F)F)ccc2NC1=O] | H | A | 1.378 | 705.0 |
| I-46 | H | CH | 3,5-(CF₃)₂ | 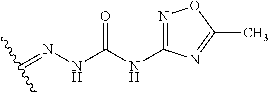 [SMILES: *=NNC(=O)Nc1noc(C)n1] | H | A | 1.062 | 531.2 |
| I-47 | H | CH | 3,5-(CF₃)₂ | N—NHC(O)NHCH₂CH₂OCH₃ | H | A | 1.085 | 507.1 |
| I-48 | H | CH | 3,5-(CF₃)₂ | N—NHC(O)NHCH(CH₃)₂ | H | A | 1.161 | 491.2 |
| I-49 | H | CH | 3,5-(CF₃)₂ | N—NHC(O)NHCH₂CF₃ | H | A | 1.162 | 531.2 |
| I-50 | H | CH | 3,5-(CF₃)₂ | 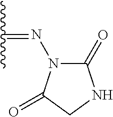 [SMILES: *=NN1C(=O)CNC1=O] | H | A | 1.044 | 489.1 |
| I-51 | H | CH | 3,5-(CF₃)₂ | N—N[C(O)N(CH₃)₂](3-Cl,4-F—C₆H₃) | H | A | 1.343 | 605.2 |
| I-52 | H | CH | 3,5-(CF₃)₂ | N—NHC(O)NHCH₂CH₂CH(CH₃)₂ | H | A | 1.268 | 519.2 |
| I-53 | H | CH | 3,5-(CF₃)₂ | 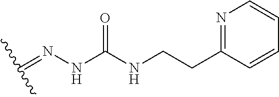 [SMILES: *=NNC(=O)NCCc1ccccn1] | CH₃ | A | 0.940 | 568.2 |
| I-54 | H | CH | 3,5-(CF₃)₂ | 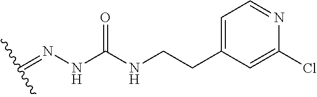 [SMILES: *=NNC(=O)NCCc1ccnc(Cl)c1] | CH₃ | A | 1.247 | 574.1 |
| I-55 | H | CH | 3,5-(CF₃)₂ | N—NHC(O)NH(4-Br—C₆H₄) | CH₃ | A | 1.377 | 618.2 |
| I-56 | H | CH | 3,5-(CF₃)₂ | N—NHC(O)NH(4-Cl—C₆H₄) | CH₃ | A | 1.364 | 573.2 |
| I-57 | H | CH | 3,5-(CF₃)₂ | 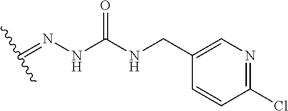 [SMILES: *=NNC(=O)NCc1ccc(Cl)nc1] | CH₃ | A | 1.218 | 588.2 |
| I-58 | H | CH | 3,5-(CF₃)₂ | N—NHC(O)NHCH₂CHF₂ | CH₃ | A | 1.179 | 527.2 |
| I-59 | H | CH | 3,5-(CF₃)₂ | 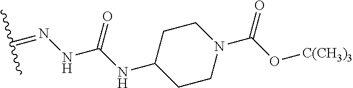 [SMILES: *=NNC(=O)NC1CCN(C(=O)OC(C)(C)C)CC1] | CH₃ | A | 1.299 | 668.3 (M + Na)⁺ |
| I-60 | H | CH | 3,5-(CF₃)₂ | 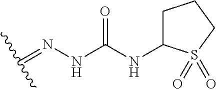 [SMILES: *=NNC(=O)NC1CCCS1(=O)=O] | CH₃ | A | 1.100 | 581.2 |
| I-61 | H | CH | 3,5-(CF₃)₂ | N—NHC(O)NHCH₂C(O)OCH₃ | CH₃ | A | 1.124 | 535.2 |
| I-62 | H | CH | 3,5-(CF₃)₂ | 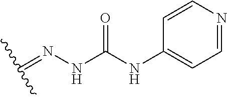 [SMILES: *=NNC(=O)Nc1ccncc1] | CH₃ | A | 0.948 | 540.2 |
| I-63 | H | CH | 3,5-(CF₃)₂ | 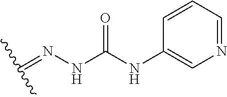 [SMILES: *=NNC(=O)Nc1cccnc1] | CH₃ | A | 0.958 | 540.2 |
| I-64 | H | CH | 3,5-(CF₃)₂ | N—NHC(O)NHCH₂(4-Cl—C₆H₄) | CH₃ | A | 1.328 | 587.2 |
| I-65 | H | CH | 3,5-(CF₃)₂ | N—NHC(O)NH(3-Cl—C₆H₄) | CH₃ | A | 1.369 | 573.2 |
| I-66 | H | CH | 3,5-(CF₃)₂ | N—NHC(O)NH—(2-Cl,4-CH₃—C₆H₃) | CH₃ | A | 1.347 | 587.2 |
| I-67 | H | CH | 3,5-(CF₃)₂ | N—NHC(O)NH—(4-SO₂CH₃—C₆H₄) | CH₃ | A | 1.159 | 617.2 |
| I-68 | H | CH | 3,5-(CF₃)₂ | N—NHC(O)NH—(4-C₆H₅—C₆H₄) | CH₃ | A | 1.412 | 615.2 |
| I-69 | H | CH | 3,5-(CF₃)₂ | N—NHC(O)NH-cC₆H₁₁ | CH₃ | A | 1.328 | 545.2 |
| I-70 | H | CH | 3,5-(CF₃)₂ | N—NHC(O)NH—(4-OCH₃—C₆H₄) | CH₃ | A | 1.260 | 569.2 |
| I-71 | H | CH | 3,5-(CF₃)₂ | N—NHC(O)NHCH₂CH₂CHSCF₃ | CH₃ | A | 1.298 | 591.2 |
| I-72 | H | CH | 3,5-(CF₃)₂ | N—OCH₂C(O)OCH₂CH₂CH₂CH₃ | CH₃ | A | 1.422 | 535.2 |
| I-73 | H | CH | 3,5-(CF₃)₂ | N—O(3-Br-C₆H₄) | CH₃ | A | 1.531 | 577.1 |
| I-74 | H | CH | 3,5-(CF₃)₂ | 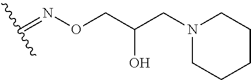 [SMILES: *=NOCC(O)CN1CCCCC1] | CH₃ | A | 0.996 | 562.2 |
| I-75 | H | CH | 3,5-(CF₃)₂ | 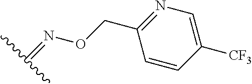 [SMILES: *=NOCc1ccc(C(F)(F)F)cn1] | CH₃ | A | 1.386 | 580.2 |
| I-76 | H | CH | 3,5-(CF₃)₂ | NOCH₂CH₂SCH₃ | CH₃ | A | 1.382 | 495.2 |
| I-77 | H | CH | 3,5-(CF₃)₂ | NOCH₂CH═CH₂ | CH₃ | A | 1.369 | 461.1 |
| I-78 | H | CH | 3,5-(CF₃)₂ | 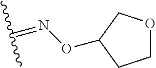 [SMILES: *=NOC1CCOC1] | CH₃ | A | 1.280 | 491.2 |
| I-79 | H | CH | 3,5-(CF₃)₂ | NOCH(COOCH₃)CH₂C₆H₅ | CH₃ | A | 1.432 | 583.2 |
| I-80 | H | CH | 3,5-(CF₃)₂ | NOCH(CH₂OH)CH(CH₃)₂ | CH₃ | A | 1.310 | 507.2 |
| I-81 | H | CH | 3,5-(CF₃)₂ | NOCH₂CHF₂ | CH₃ | A | 1.319 | 485.1 |
| I-82 | H | CH | 3,5-(CF₃)₂ | 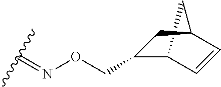 [SMILES: *=NOC[C@@H]1C[C@@H]2C=C[C@H]1C2] | CH₃ | A | 1.564 | 527.3 |
| I-83 | H | N | 3-(1-CN-cC₃H₅), | NOCH₃ | NH₂ | B | 1.488 | 434.1 |
| | | | 5-CF₃ | | | | | |
| I-84 | H | CH | 3,5-(CF₃)₂ | 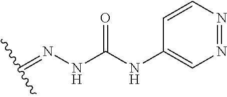 [SMILES: *=NNC(=O)Nc1ccnnc1] | CH₃ | A | 0.969 | 541.2 |
| I-85 | H | CH | 3,5-(CF₃)₂ | 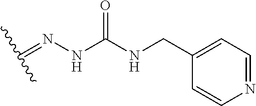 [SMILES: *=NNC(=O)NCc1ccncc1] | CH₃ | A | 0.935 | 554.3 |
| I-86 | H | CH | 3,5-(CF₃)₂ | N—NHC(O)NHCH₂C(O)NHCH(CH₃)₂ | CH₃ | A | 1.098 | 562.3 |
| I-87 | H | CH | 3,5-(CF₃)₂ | N—NHC(O)NHCH₂CH═CCl₂ | CH₃ | A | 1.314 | 571.2 |
| I-88 | H | CH | 3,5-(CF₃)₂ | N—NHC(O)NHCH₂C(O)NHcC₃H₅ | CH₃ | A | 1.066 | 560.3 |
| I-89 | H | CH | 3,5-(CF₃)₂ | 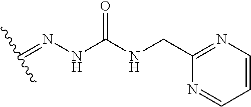 [SMILES: *=NNC(=O)NCc1ncccn1] | CH₃ | A | 1.074 | 555.3 |
| I-90 | H | CH | 3,5-(CF₃)₂ | N—NHC(O)NHCH₂CH(CH₃)₂ | CH₃ | A | 1.259 | 519.2 |
| I-91 | H | CH | 3,5-(CF₃)₂ | 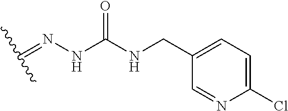 [SMILES: *=NNC(=O)NCc1ccc(Cl)nc1] | CH₃ | A | 1.305 | 574.2 |
| I-92 | H | CH | 3,5-(CF₃)₂ | 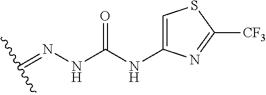 [SMILES: *=NNC(=O)Nc1csc(C(F)(F)F)n1] | CH₃ | A | 1.333 | 614.2 |
| I-93 | H | CH | 3,5-(CF₃)₂ | 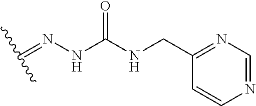 [SMILES: *=NNC(=O)NCc1ccncn1] | CH₃ | A | 1.064 | 555.2 |
| I-94 | H | CH | 3,5-(CF₃)₂ | 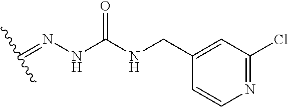 [SMILES: *=NNC(=O)NCc1ccnc(Cl)c1] | CH₃ | A | 1.193 | 588.2 |
| I-95 | H | CH | 3,5-(CF₃)₂ | 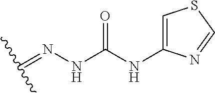 [SMILES: *=NNC(=O)Nc1cscn1] | CH₃ | A | 1.183 | 546.2 |
| I-96 | H | CH | 3,5-(CF₃)₂ | 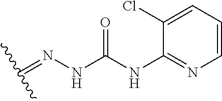 [SMILES: *=NNC(=O)Nc1ncccc1Cl] | CH₃ | A | 1.265 | 574.2 |
| I-97 | H | CH | 3,5-(CF₃)₂ | 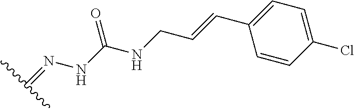 [SMILES: *=NNC(=O)NC/C=C/c1ccc(Cl)cc1] | CH₃ | A | 1.367 | 613.2 |
| I-98 | H | CH | 3,5-(CF₃)₂ | 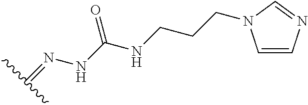 [SMILES: *=NNC(=O)NCCCn1ccnc1] | CH₃ | A | 0.943 | 571.3 |
| I-99 | H | CH | 3,5-(CF₃)₂ | 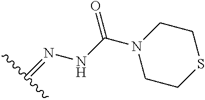 [SMILES: *=NNC(=O)N1CCSCC1] | CH₃ | A | 1.224 | 549.2 |
| I-100 | H | CH | 3,5-(CF₃)₂ | N—OCH₂cC₄H₇ | H | A | 1.421 | 475.2 |
| I-101 | H | CH | 3,5-(CF₃)₂ | N—O(3,4-Cl₂—C₆H₃) | H | A | 1.499 | 551.2 |
| I-102 | H | CH | 3,5-(CF₃)₂ | N—OCH₂CN | H | A | 1.171 | 446.1 |
| I-103 | H | CH | 3,5-(CF₃)₂ | N—OCH₂(1-CH₃—cC₃H₅) | H | A | 1.404 | 475.2 |
| I-104 | H | CH | 3,5-(CF₃)₂ | 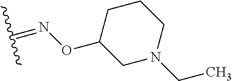 [SMILES: *=NOC1CCCN(CC)C1] | H | A | 0.970 | 518.3 |
| I-105 | H | CH | 3,5-(CF₃)₂ | N—OCH₂CF₂CF₂H | H | A | 1.301 | 521.2 |
| I-106 | H | CH | 3,5-(CF₃)₂ | N—OCH₂CH₂cC₃H₅ | H | A | 1.398 | 475.2 |
| I-107 | H | CH | 3,5-(CF₃)₂ | 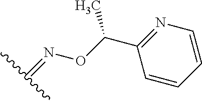 [SMILES: *=NO[C@H](C)c1ccccn1] | H | A | 1.021 | 512.2 |
| I-108 | H | CH | 3,5-(CF₃)₂ | N—OCH₂(2-F,3-CF₃—C₆H₃) | H | A | 1.433 | 583.2 |
| I-109 | H | CH | 3,5-(CF₃)₂ | N—OCH₂[2,4-(OCH₃)₂-C₆H₃] | H | A | 1.375 | 579.2 |
| | | | | | | | | (M + |
| | | | | | | | | Na)⁺ |
| I-110 | H | CH | 3,5-(CF₃)₂ | N—O(4-CN—C₆H₄) | H | A | 1.322 | 508.2 |
| I-111 | H | CH | 3,5-(CF₃)₂ | 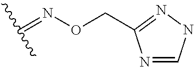 [SMILES: *=NOCc1nc[nH]n1] | H | A | 1.029 | 488.1 |
| I-112 | H | CH | 3,5-(CF₃)₂ | N—OCH₂CH₂CH₂CF₃ | H | A | 1.349 | 517.2 |
| I-113 | H | CH | 3,5-(CF₃)₂ | 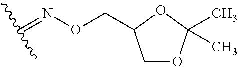 [SMILES: *=NOCC1COC(C)(C)O1] | H | A | 1.274 | 521.2 |
| I-114 | H | CH | 3,5-(CF₃)₂ | N—OCH(CH₃)CN | H | A | 1.225 | 460.1 |
| I-115 | H | CH | 3,5-(CF₃)₂ | N—NHC(O)NHCH₂CN | H | A | 1.107 | 487.1 |
| I-116 | H | CH | 3,5-(CF₃)₂ | N—N(CH₃)C(O)NHC₆H₅ | H | A | 1.314 | 539.2 |
| I-117 | H | CH | 3,5-(CF₃)₂ | 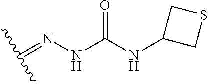 [SMILES: *=NNC(=O)NC1CSC1] | H | A | 1.179 | 521.2 |
| I-118 | H | CH | 3,5-(CF₃)₂ | N—N[CH(CH₃)₂]C(O)NHC₆H₅ | H | A | 1.388 | 567.3 |
| I-119 | H | CH | 3,5-(CF₃)₂ | 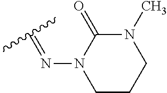 [SMILES: *=NN1CCCN(C)C1=O] | H | A | 1.148 | 503.2 |
| I-120 | H | CH | 3,5-(CF₃)₂ | 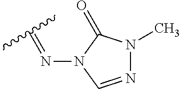 [SMILES: *=Nn1cnn(C)c1=O] | H | A | 1.108 | 488.2 |
| I-121 | H | CH | 3,5-(CF₃)₂ | 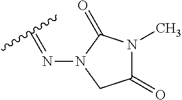 [SMILES: *=NN1CC(=O)N(C)C1=O] | H | A | 1.109 | 503.2 |
| I-122 | H | CH | 3,5-(CF₃)₂ | N—N(C₆H₅)C(O)NHC₆H₅ | H | A | 1.371 | 601.2 |
| I-123 | H | CH | 3,5-(CF₃)₂ | N—N(C₆H₅)C(O)NHCH₃ | H | A | 1.211 | 539.2 |
| I-124 | H | CH | 3,5-(CF₃)₂ | N—NHC(O)NHCH₂CH═CCl₂ | H | A | 1.253 | 558.2 |
| I-125 | H | CH | 3,5-(CF₃)₂ | N—NHC(O)NHCH₂CH₂SCF₃ | H | A | 1.247 | 577.2 |
| I-126 | H | CH | 3,5-(CF₃)₂ | N—NHC(O)N(CH₃)C₂H₅ | H | A | 1.123 | 491.2 |
| I-127 | H | CH | 3,5-(CF₃)₂ | N—N(CH₃)C(O)NHCH(CH₃)₂ | H | A | 1.236 | 505.2 |
| I-128 | H | CH | 3,5-(CF₃)₂ | N—NHC(O)NHCH₂CH₂CH₂CH₃ | H | A | 1.215 | 505.2 |
| I-129 | H | CH | 3,5-(CF₃)₂ | N—NHC(O)NHCH₂CH₃ | H | A | 1.120 | 477.2 |
| I-130 | H | CH | 3,5-(CF₃)₂ | 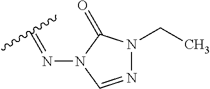 [SMILES: *=Nn1cnn(CC)c1=O] | H | A | 1.162 | 502.2 |
| I-131 | H | N | 3-(1-CN-cC₃H₅), | NOCH₃ | CH₃ | B | 1.621 | 433.1 |
| | | | 5-CF₃ | | | | | |
| I-132 | H | CH | 3-Cl, 5-(SO₂CH₃) | NOCH₃ | CH₃ | B | 1.413 | 411.1 |
| I-133 | H | CH | 3,5-(CF₃)₂ | NOCH₂C═CH | H | A | 1.236 | 445.1 |
| I-134 | H | CH | 3,5-(CF₃)₂ | N—OCH(CH₂OCH₃)₂ | H | A | 1.231 | 509.2 |
| I-135 | H | CH | 3,5-(CF₃)₂ | N—OCH(CH₃)(3-CF₃—C₆H₄) | H | A | 1.459 | 579.2 |
| I-136 | H | CH | 3,5-(CF₃)₂ | 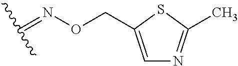 [SMILES: *=NOCc1cnc(C)s1] | H | A | 1.192 | 518.1 |
| I-137 | H | CH | 3,5-(CF₃)₂ | N—OCH₂(2-C₆H₅—C₆H₄) | H | A | 1.495 | 573.2 |
| I-138 | H | CH | 3,5-(CF₃)₂ | N—OCH₂CH₂O[3,5-(CH₃)₂—C₆H₃] | H | A | 1.448 | 555.2 |
| I-139 | H | CH | 3,5-(CF₃)₂ | N—OCH₂CH(CH₃)O(3-Cl—C₆H₄) | H | A | 1.464 | 575.2 |
| I-140 | H | CH | 3,5-(CF₃)₂ | N—OCH(CH₃)CN | H | A | 1.292 | 459.1 |
| I-141 | H | CH | 3,5-(CF₃)₂ | N—OCH₂(3-OC₂H₅—C₆H₄) | H | A | 1.404 | 541.2 |
| I-142 | H | CH | 3,5-(CF₃)₂ | N—OCH₂CH(CH₃)C₆H₅ | H | A | 1.450 | 525.2 |
| I-143 | H | CH | 3,5-(CF₃)₂ | 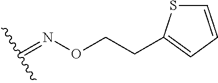 [SMILES: *=NOCCc1cccs1] | H | A | 1.389 | 517.1 |
| I-144 | H | CH | 3,5-(CF₃)₂ | 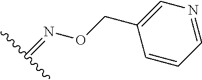 [SMILES: *=NOCc1cccnc1] | H | A | 0.973 | 498.2 |
| I-145 | H | CH | 3,5-(CF₃)₂ | N—OCH(C₂H₅)(4-Cl—C₆H₄) | H | A | 1.511 | 559.2 |
| I-146 | H | CH | 3,5-(CF₃)₂ | 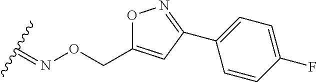 [SMILES: *=NOCc1cc(-c2ccc(F)cc2)no1] | H | A | 1.384 | 582.2 |
| I-147 | H | CH | 3,5-(CF₃)₂ | N—OCH₂(4-OCH₃—C₆H₄) | H | A | 1.349 | 527.2 |
| I-148 | H | CH | 3,5-(CF₃)₂ | 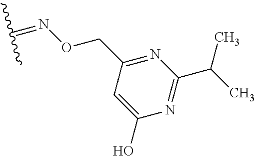 [SMILES: *=NOCc1cc(O)nc(C(C)C)n1] | CH₃ | A | 1.186 | 571.2 |
| I-149 | H | CH | 3,5-(CF₃)₂ | N—OCH₂CH₂CH₂CH₂(4-CH₃—C₆H₄) | CH₃ | A | 1.588 | 567.3 |
| I-150 | H | CH | 3,5-(CF₃)₂ | 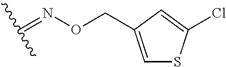 [SMILES: *=NOCc1csc(Cl)c1] | CH₃ | A | 1.474 | 551.1 |
| I-151 | H | CH | 3,5-(CF₃)₂ | 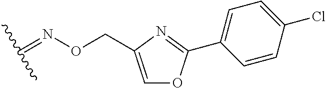 [SMILES: *=NOCc1coc(-c2ccc(Cl)cc2)n1] | CH₃ | A | 1.473 | 612.2 |
| I-152 | H | CH | 3,5-(CF₃)₂ | 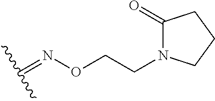 [SMILES: *=NOCCN1CCCC1=O] | CH₃ | A | 1.191 | 532.2 |
| I-153 | H | CH | 3,5-(CF₃)₂ | N—OCH(CH₃)[2,5(CH₃)₂—C₆H₃] | CH₃ | A | 1.550 | 553.3 |
| I-154 | H | CH | 3,5-(CF₃)₂ | N—OCH(CH₃)(2-CF₃—C₆H₄) | CH₃ | A | 1.527 | 593.2 |
| I-155 | H | CH | 3,5-(CF₃)₂ | N—OCH(CH₃)(4-OCF₂CHF₂—C₆H₄) | CH₃ | A | 1.490 | 641.2 |
| I-156 | H | CH | 3,5-(CF₃)₂ | N—OCH₂CH(OH)CH₃ | CH₃ | A | 1.183 | 479.2 |
| I-157 | H | CH | 3,5-(CF₃)₂ | N—OCH₂CH₂N(CH₃)CH₂C₆H₅ | CH₃ | A | 1.058 | 568.2 |
| I-158 | H | CH | 3,5-(CF₃)₂ | N—OCH(CH₃)[3,4(OCH₃)₂—C₆H₃] | CH₃ | A | 1.391 | 585.2 |
| I-159 | H | CH | 3,5-(CF₃)₂ | N—OCH₂CH₂N(CH₃)₂ | CH₃ | A | 0.968 | 492.2 |
| I-160 | H | CH | 3,5-(CF₃)₂ | 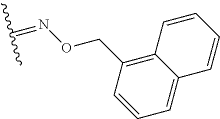 [SMILES: *=NOCc1cccc2ccccc12] | CH₃ | A | 1.501 | 561.2 |
| I-161 | H | CH | 3,5-(CF₃)₂ | NOC₆H₅ | CH₃ | A | 1.434 | 497.2 |
| I-162 | H | CH | 3,5-(CF₃)₂ | N—OCH₂CH₂CH₂O(3-F—C₆H₄) | CH₃ | A | 1.480 | 573.2 |
| I-163 | H | CH | 3,5-(CF₃)₂ | N—OCH₂CH(C₂H₅)O(4-F—C₆H₄) | CH₃ | A | 1.515 | 587.2 |
| I-164 | H | CH | 3,5-(CF₃)₂ | NOC(CH₃)₃ | CH₃ | A | 1.472 | 477.1 |
| I-165 | CH₂cC₃H₅ | N | 3-SO₂—(4-F— | NOCH₃ | CH₃ | B | 1.753 | 580.2 |
| | | | C₆H₄), 5-CF₃ | | | | | |
| I-166 | H | N | 3-(SO₂CH₃), 5-CF₃ | NOCH₃ | NH₂ | B | 1.292 | 447.2 |
| I-167 | H | CH | 3,5-(CF₃)₂ | NOCH₃ | NH₂ | B | 1.686 | 461.2 |
| I-168 | CH₂cC₅H₉ | CH | 3-(1-CN-cC₃H₅), | NOCH₃ | NH₂ | B | 1.927 | 515.3 |
| | | | 5-CF₃ | | | | | |
| I-169 | H | CH | 3,5-(CF₃)₂ | NOCH₃ | N(CH₃) | B | 1.791 | 478.2 |
| | | | | | C₂H₅ | | | |
| I-170 | H | CH | 3,5-(CF₃)₂ | NOH | N(CH₃) | B | 1.521 | 464.2 |
| | | | | | C₂H₅ | | | |
| I-171 | H | CH | 3,5-(CF₃)₂ | NOCH₃ | NHCH₂ | B | 1.689 | 475.2 |
| | | | | | CN | | | |
| I-172 | H | CH | 3,5-(CF₃)₂ | NOCH₃ | SCH₃ | B | 1.834 | 433.2 |
| I-173 | CH₂cC₃H₅ | CH | 3,5-(CF₃)₂ | NOCH₃ | SCH₃ | B | 1.979 | 487.2 |
| I-174 | H | CH | 3-Cl, 5-CF₃ | NOCH₃ | NH₂ | A | 1.143 | 402.2 |
| I-175 | H | N | 3-Cl, 5-C(CH₃)₃ | NOCH₃ | NH₂ | A | 1.184 | 391.3 |
| I-176 | H | CH | 3-Cl, 5-SCH₃ | NOCH₃ | NH₂ | A | 1.096 | 390.2 |
| I-177 | H | CH | 3,4-F₂, 5-CN | NOCH₃ | NH₂ | A | 0.96 | 361.2 |
| I-178 | H | CH | 3-(1-SO₂CH₃)cC₃H₄ | NOCH₃ | NH₂ | A | 0.834 | 418.2 |
| I-179 | H | CH | 3,5-Cl₂, 4-F | NOCH₃ | NH₂ | A | 1.121 | 386.1 |
| I-180 | H | N | 3,5-Cl₂ | NOCH₃ | NH₂ | A | 1.003 | 369.1 |
| I-181 | H | CH | 3-Cl, 5-SO₂CF₃ | NOCH₃ | NH₂ | A | 1.149 | 466.2 |
| I-182 | H | CH | 3,4,5-F₃ | NOCH₃ | NH₂ | A | 1.003 | 354.2 |
| I-183 | H | CH | 3-Br, 5-CH(CH₃)₂ | NOCH₃ | NH₂ | A | 1.217 | 421.7 |
| I-184 | H | CH | 3-Br, 5-OCH(CH₃)₂ | NOCH₃ | NH₂ | A | 1.191 | 437.7 |
| I-185 | H | N | 3-OcC₃H₅ | NOCH₃ | NH₂ | A | 0.896 | 357.3 |
| I-186 | H | CH | 3-S(3-Cl,5-CF₃)C₆H₃ | NOCH₃ | NH₂ | A | 1.362 | 510.2 |
| I-187 | H | CH | 3-O(3-Cl,5-CF₃)C₆H₃ | NOCH₃ | NH₂ | A | 1.298 | 494.2 |
| I-188 | H | CH | 3-SCF₃ | NOCH₃ | NH₂ | A | 1.131 | 400.2 |
| I-189 | H | CH | 3,5-Br₂ | NOCH₃ | NH₂ | A | 1.167 | 458.1 |
| I-190 | H | CH | 3,5-F₂, 4-CN | NOCH₃ | NH₂ | A | 0.969 | 361.2 |
| I-191 | H | CH | 3-CF₃, 4-CH₃ | NOCH₃ | NH₂ | A | 1.11 | 381.9 |
| I-192 | H | CH | 3-CF₃, 5-C(CH₃)₂CN | NOCH₃ | NH₂ | A | 1.088 | 435.1 |
| I-193 | H | CH | 3-(2,2-F₂-CC₃H₃) | NOCH₃ | NH₂ | A | 1.000 | 376.1 |
| I-194 | H | CH | 3-Br, 5-OC(CH₃)₃ | NOCH₃ | NH₂ | A | 1.191 | 452.0 |
| I-195 | H | CH | 3-Cl, 5-cC₃H₅ | NOCH₃ | NH₂ | A | 1.123 | 374.0 |
| I-196 | H | CH | 3-Cl, 5-CHF₂ | NOCH₃ | NH₂ | A | 1.044 | 384.0 |
| I-197 | H | CH | 3-Br, 5-OcC₃H₅ | NOCH₃ | NH₂ | A | 1.137 | 436.0 |
| I-198 | H | CH | 3-CHCF₂ | NOCH₃ | NH₂ | A | 1.018 | 362.0 |
| I-199 | H | CH | 3-Br, 5-OCF₃ | NOCH₃ | NH₂ | A | 1.177 | 463.9 |
| I-200 | H | CH | 3-Br, 5-OCHF₂ | NOCH₃ | NH₂ | A | 1.086 | 446.0 |
| I-201 | H | N | 3-O(4-Br-C₆H₄), 5-Cl | NOCH₃ | NH₂ | A | 1.232 | 507.0 |
| I-202 | H | CH | 3,5-(CH₃)₂ | NOCH₃ | NH₂ | A | 1.007 | 328.1 |
| I-203 | H | CH | 3-CHF₂ | NOCH₃ | NH₂ | A | 0.937 | 350.0 |
| I-204 | H | CH | 3-Br, 5-CH₃ | NOCH₃ | NH₂ | A | 1.072 | 392.0 |
| I-205 | H | CH | 3-cC₃H₅, 5-F | NOCH₃ | NH₂ | A | 1.054 | 358.1 |
| I-206 | H | CH | 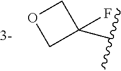 [SMILES: *C1(F)COC1] | NOCH₃ | NH₂ | A | 0.877 | 374.1 |
| I-207 | H | CH | 3,5-Cl₂ | NOCH₃ | NH₂ | A | 1.101 | 368.0 |
| I-208 | H | CF | 3-Cl | NOCH₃ | NH₂ | A | 0.997 | 352.0 |
| I-209 | H | CH | 3-(1-OCH₃)-cC₄H₆ | NOCH₃ | NH₂ | A | 1.005 | 384.2 |
| I-210 | H | CH | 3-CF₃, 5-S(O)₂CH₃ | NOCH₃ | NH₂ | A | 0.952 | 446.0 |
| I-211 | H | CH | 3-(1-CN)-cC₃H₄ | NOCH₃ | NH₂ | A | 0.918 | 365.1 |
| I-212 | H | CH | 3-Cl, 5-CN | NOCH₃ | NH₂ | A | 0.979 | 359.0 |
| I-213 | H | CH | 3-Br, 5-CN | NOCH₃ | NH₂ | A | 0.997 | 405.0 |
| I-214 | H | CH | 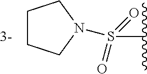 [SMILES: *S(=O)(=O)N1CCCC1] | NOCH₃ | NH₂ | A | 0.932 | 433.1 |
| I-215 | H | N | 3-Br | NOCH₃ | NH₂ | A | 0.861 | 380.9 |
| I-216 | H | CH | 3-Br, 5-CF₃ | NOCH₃ | NH₂ | A | 1.150 | 448.0 |
| I-217 | H | CBr | 3,5-Cl₂ | NOCH₃ | NH₂ | A | 1.176 | 448.0 |
| I-218 | H | CH | 3-C(CH₃)₃ | NOCH₃ | NH₂ | A | 1.110 | 356.1 |
| I-219 | H | CH | 3-Cl, 5-S(O)₂CH₃ | NOCH₃ | NH₂ | A | 0.898 | 412.1 |
| I-220 | H | N | 3-OCHF₂ | NOCH₃ | NH₂ | A | 0.944 | 367.0 |
| I-221 | H | CF | 3-Br | NOCH₃ | NH₂ | A | 1.012 | 397.9 |
| I-222 | H | CF | 3-F | NOCH₃ | NH₂ | A | 0.930 | 336.0 |
| I-223 | H | CCl | 3-CF₃ | NOCH₃ | NH₂ | A | 1.106 | 402.0 |

### Biological Examples

If not otherwise specified, the test solutions were prepared as follow:

The active compound was dissolved at the desired concentration in a mixture of 1:1 (vol:vol) distilled water:acetone. The test solution was prepared on the day of use.

The activity of the compounds of formula I of the present invention can be demonstrated and evaluated by the following biological tests.

B.1 Green Peach Aphid (Myzus persicae)

For evaluating control of green peach aphid (Myzus persicae) through systemic means, the test unit consisted of 96-well-microtiter plates containing liquid artificial diet under an artificial mem brane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds I-3, I-4, I-5, I-7, I-8, I-9, I-12, I-13, I-14, I-15, I-18, I-19, I-21, I-35, I-50, I-83, I-93, I-99, I-120, I-121, I-123, I-126, I-127, I-130, I-131, I-132, I-152, I-159, I-169, I-170, I-174, I-176, and I-180, resp., at 2500 ppm showed at least 75% mortality in comparison with untreated controls.

B.2 Tobacco Budworm (Heliothis virescens)

For evaluating control of tobacco budworm (Heliothis virescens), the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality was then visually assessed.

In this test, compounds I-1, I-3, I-5, I-7, I-10, I-11, I-12, I-13, I-14, I-15, I-16, I-19, I-21, I-22, I-23, I-24, I-30, I-33, I-35, I-53, I-61, I-64, I-75, I-76, I-77, I-78, I-81, I-82, I-83, I-87, I-89, I-90, I-92, I-93, I-95, I-102, I-105, I-111, I-112, I-113, I-114, I-120, I-121, I-123, I-125, I-127, I-129, I-130, I-131, I-133, I-136, I-140, I-144, I-148, I-150, I-151, I-155, I-157, I-158, I-159, I-167, I-169, I-170, I-171, I-172, I-174, I-180, and I-181, resp., at 2500 ppm showed at least 75% mortality in comparison with untreated controls.

B.3 Boll Weevil (Anthonomus grandis)

For evaluating control of boll weevil (Anthonomus grandis), the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality was then visually assessed.

In this test, compounds I-1, I-3, I-4, I-5, I-7, I-8, I-10, I-11, I-12, I-13, I-14, I-15, I-16, I-17, I-18, I-19, I-21, I-22, I-23, I-24, I-30, I-34, I-35, I-39, I-40, I-41, I-46, I-48, I-50, I-53, I-58, I-59, I-60, I-61, I-64, I-71, I-74, I-75, I-76, I-78, I-83, I-85, I-87, I-88, I-89, I-90, I-93, I-95, I-99, I-102, I-103, I-104, I-107, I-108, I-111, I-112, I-114, I-115, I-117, I-118, I-120, I-121, I-122, I-123, I-125, I-126, I-127, I-128, I-129, I-130, I-131, I-132, I-133, I-134, I-135, I-136, I-140, I-142, I-144, I-147, I-148, I-150, I-155, I-157, I-158, I-159, I-160, I-162, I-167, I-169, I-170, I-171, I-172, I-174, I-176, I-179, I-180, I-181, I-183, I-184, and I-188, resp., at 2500 ppm showed at least 75% mortality in comparison with untreated controls.

B.4. Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A non-ionic surfactant (Kinetic®) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an auto-mated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants.

In this test, compounds I-1, I-4, I-5, I-7, I-9, I-10, I-11, I-12, I-13, I-14, I-15, I-16, I-17, I-18, I-19, I-21, I-22, I-23, I-24, I-30, I-41, I-46, I-47, I-57, I-64, I-75, I-76, I-77, I-78, I-81, I-82, I-83, I-87, I-89, I-93, I-95, I-100, I-102, I-103, I-104, I-106, I-107, I-111, I-112, I-113, I-114, I-115, I-120, I-121, I-122, I-123, I-125, I-126, I-127, I-128, I-129, I-130, I-131, I-133, I-134, I-135, I-136, I-139, I-140, I-144, I-145, I-148, I-150, I-151, I-155, I-156, I-157, I-159, I-167, I-171, and I-172, resp., at 300 ppm at least 75% mortality in comparison with untreated controls.

B.5 Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 28±1° C., 80±5% RH for 2 days. Larval mortality was then visually assessed.

In this test, compounds I-1, I-2, I-3, I-4, I-5, I-7, I-8, I-9, I-10, I-11, I-12, I-13, I-14, I-15, I-16, I-17, I-18, I-19, I-21, I-22, I-23, I-24, I-25, I-26, I-30, I-33, I-35, I-38, I-39, I-40, I-41, I-42, I-43, I-44, I-46, I-47, I-48, I-49, I-50, I-51, I-55, I-56, I-57, I-59, I-62, I-64, I-69, I-70, I-71, I-74, I-75, I-76, I-77, I-78, I-79, I-81, I-82, I-83, I-85, I-87, I-90, I-91, I-92, I-93, I-94, I-95, I-97, I-99, I-100, I-102, I-103, I-104, I-105, I-107, I-111, I-112, I-113, I-114, I-115, I-117, I-118, I-119, I-120, I-121, I-122, I-123, I-124, I-125, I-126, I-127, I-128, I-129, I-130, I-131, I-132, I-133, I-134, I-135, I-136, I-140, I-144, I-145, I-148, I-150, I-151, I-153, I-154, I-155, I-157, I-158, I-159, I-160, I-161, I-163, I-167, I-169, I-170, I-171, I-172, I-173, I-174, I-176, I-180, I-181, I-183, I-186, and I-187, resp., at 2500 ppm showed at least 75% mortality in comparison with untreated controls.

1. A compound of formula I 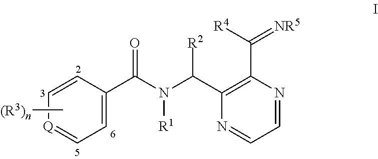
wherein
R¹ is H, OH, C₁-C₆-alkyl, C₁-C₆-haloalkyl, C₃-C₆-cycloalkyl, C₃-C₆-halocycloalkyl, C₁-C₅-alkoxy, C₂-C₄-alkenyl, C₂-C₄-alkynyl, C₁-C₄-alkyl-C₃-C₆-cycloalkyl, C₁-C₄-alkyl-C₃-C₆-halocycloalkyl, which groups are unsubstituted, or partially or fully substituted with R¹¹;
or C(═N—R¹¹)R¹², C(O)R^{11a};
R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkylC1-C₂-alkyl which ring is unsubstituted or substituted with 1 or 2 halogen; 3- to 6-membered heterocyclyl, 5- or 6-membered hetaryl, phenyl, or phenoxy, naphthyl, naphthoxy, which rings are unsubstituted or substituted with R^{3a};
R^{11a} is NR¹²R¹³, C(O)NH₂, C(S)NH₂, C(O)OH, OR¹⁴, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with halogen, C₁-C₃-haloalkyl, and/or CN;
R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl, S(O)ₘ—NR¹²¹R¹³¹;
3- to 6-membered heterocyclyl, 5- or 6-membered hetaryl, phenyl, phenoxy, phenyl-C₁-C₄-alkyl, naphthyl, naphthoxy, which rings are unsubstituted or substituted with R^{3a}; or
R¹² and R¹³, together with the nitrogen atom to which they are bound, form a 3-, 4-, 5-, 6- or 7-membered saturated, partially unsaturated or fully unsaturated heterocycle, which heterocycle may additionally contain 1 or 2 heteroatoms or heteroatom groups selected from N, O, and S(O)ₘ as ring members, and which heterocycle is unsubstituted or substituted with one or more substituents selected from halogen, C₁-C₄-alkyl, C₁-C₄-haloalkyl, C₁-C₄-alkoxy, C₁-C₄-haloalkoxy, and oxo;
R¹²¹ and R¹³¹ are independently from each other hydrogen, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl or 5- or 6-membered hetaryl which rings are unsubstituted or substituted with R^{3a}; or
R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with halogen, C₁-C₃-haloalkyl, and/or CN;
m is 0, 1, or 2;
R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R₃ₐ;
R² is CN, C₁-C₃-alkyl, C₁-C₃-haloalkyl, C₂-C₃-alkynyl;
R³ is halogen, CN, NO₂, C₁-C₄-alkyl, C₃-C₆-cycloalkyl, C₁-C₆-haloalkyl, C₁-C₆-halocycloalkyl, OR¹⁴, S(O)ₘ—R¹⁴; which are unsubstituted or substituted with R^{3a};
R^{3a} halogen, CN, NO₂, OR¹⁵, C₁-C₄-alkyl, C₁-C₄-haloalkyl, C₁-C₄-alkoxy-C₁-C₄-alkyl, C₃-C₆-cycloalkyl, C₃-C₆-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, phenyl, phenyl-C₁-C₄-alkyl, which rings are unsubstituted or substituted with halogen, CN, NO₂, C₁-C₄-alkyl, C₁-C₄-haloalkyl, C₁-C₄-alkoxy-C₁-C₄-alkyl, C₁-C₄-alkoxy, C₁-C₄-haloalkoxy,
R^{3a} as substituent of cycloalkyl or heterocyclyl may also denote oxo;
n is 0, 1, 2, or 3;
Q is CH, CR³, or N;
R⁴ is H, OH, CN, C₁-C₄-alkyl, C₃-C₆-cycloalkyl, C₁-C₆-haloalkyl, C₁-C₆-halocycloalkyl, C₂-C₄-alkenyl, C₂-C₄-haloalkenyl, C₂-C₄-alkynyl, C₁-C₄-alkoxy, C₁-C₄-haloalkoxy, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, NR¹²R¹³, C(O)NR¹²R¹³, C(O)OR¹⁴, 3- to 6-membered heterocyclyl, 5- or 6-membered hetaryl, or phenyl, which rings are unsubstituted or substituted with R³;
R⁵ is H, OR¹⁵, NR¹²R¹³, or C₁-C₆-alkyl which is unsubstituted, or partially or fully substituted with R¹¹;
R¹⁵ is H, C₁-C₄-alkyl, or C₁-C₄-haloalkyl, C₃-C₇-cycloalkyl, C₃-C₇-halocycloalkyl, C₃-C₇-cycloalkenyl, C₃-C₇-halocycloalkenyl, which carbon chains or rings are unsubstituted or partially or fully substituted with R¹¹; or 3- to 6-membered heterocyclyl, 5- or 6-membered hetaryl, or phenyl, which rings are unsubstituted or substituted with R³;
and N-oxides, stereoisomers, and agriculturally or veterinarily acceptable salts thereof.

2. The compound of formula I according to claim 1, wherein
R¹¹ is CN, NO₂, NR¹²R¹³, C(O)NH₂, C(S)NH₂, C(O)OH, OR¹⁴, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NR¹²¹R¹³¹, S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl, which rings are unsubstituted or substituted with halogen, C₁-C₃-haloalkyl, and/or CN;
R¹²¹ and R¹³¹ are independently from each other hydrogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy; C₁-C₄-alkyl-phenyl, C₁-C₄-alkyl-3-6-membered hetaryl, phenyl, 3- to 6-membered heterocyclyl or 5- or 6-membered hetaryl which rings are unsubstituted or substituted with halogen, C₁-C₃-haloalkyl, and/or CN; or
R¹²¹ and R¹³¹ together with the nitrogen atom they are bound to form a 3-6 membered saturated, partially or fully unsaturated heterocycle, which may further contain 1 or 2 heteroatoms ring members selected from N, O and S, wherein S may be oxidized, which heterocycle is unsubstituted or substituted with halogen, C₁-C₃-haloalkyl, and/or CN;
R^{3a} halogen, CN, NO₂, C₁-C₄-alkyl, C₁-C₄-haloalkyl, C₁-C₄-alkoxy-C₁-C₄-alkyl, C₁-C₄-haloalkoxy, C₃-C₄-cycloalkyl, C₃-C₄-halocycloalkyl, S(O)ₘC₁-C₄-alkyl, S(O)ₘ—C₁-C₄-haloalkyl, S(O)ₘ—C₃-C₄-cycloalkyl, S(O)ₘ—C₃-C₄-halocycloalkyl;
R¹⁴ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R³;
R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, 5- or 6-membered hetaryl, or phenyl, which rings are unsubstituted or substituted with R³;
and N-oxides, stereoisomers, and agriculturally or veterinarily acceptable salts thereof.

3. The compound of formula I according to claim 1, wherein R¹ is H or CH₂-cC₃H₅.

4. The compound of formula I according to claim 1, wherein R² is CH₃.

5. The compound of formula I according to claim 1, wherein R³ is halogen, CN, C₁-C₄-haloalkyl, C₁-C₄-haloalkoxy, C₃-C₄-cycloalkyl unsubstituted or substituted with one or more CN, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or S(O)ₘ—R¹⁴, wherein R¹⁴ is phenyl, which is partially substituted with R^{3a}.

6. The compound of formula I according to claim 1, wherein n is 2 and R³ is in positions 3 and 5.

7. The compound of formula I according to claim 1, wherein Q is CH or CR³.

8. The compound of formula I according to claim 1, wherein R⁴ is CH₃ or NH₂.

9. The compound of formula I according to claim 1, wherein R⁵ is OH, OCH₃, OCH₂CF₃, or OCH₂—C₆H₅.

10. The compound of formula I according to claim 1, which is mainly isomer I.A.

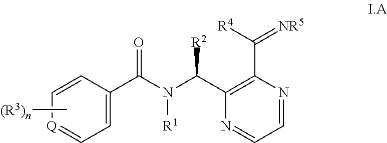

11. An intermediate compound of formula III wherein the variables are as defined for formula I in claim 1.

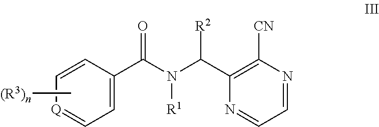

12. An agricultural or veterinary composition comprising at least one compound according to claim 1 and/or at least one agriculturally or veterinarily acceptable salt thereof, and at least one inert liquid and/or solid agriculturally or veterinarily acceptable carrier.

13. An agricultural composition for combating animal pests comprising at least one compound as defined in claim 1 and at least one inert liquid and/or solid acceptable carrier and, optionally, at least one surfactant.

14. A method for combating or controlling invertebrate pests, comprising contacting said pest or its food supply, habitat or breeding grounds with a pesticidally effective amount of at least one compound as defined in claim 1.

15. A method for protecting growing plants from attack or infestation by invertebrate pests, comprising contacting a plant, or soil or water in which the plant is growing, with a pesticidally effective amount of at least one compound as defined in claim 1.

16. A seed comprising a compound as defined in claim 1, or the enantiomers, diastereomers, or salts thereof, in an amount of from 0.1 g to 10 kg per 100 kg of seed.

17. A method for treating or protecting an animal from infestation or infection by invertebrate pests comprising bringing the animal in contact with a pesticidally effective amount of at least one compound of the formula I as defined in claim 1, a stereoisomer thereof and/or at least one veterinarily acceptable salt thereof.
